(19) United States
(12) Patent Application Publication
(10) Pub. No.: US 2005/0037960 A1
(43) Pub. Date: Feb. 17, 2005

(54) Methods and compositions for sealing tissue leaks

(76) Inventors: James Rolke, Fitzwilliam, NH (US); Luis Burzio, Mountain Top, PA (US)

Correspondence Address: Dr. James Wilkie;Surgical Sealants, Inc., 150 New Boston Street, Woburn, MA, 01801, US

(21) Appl. No.: 10/675,460

(22) Filed: Sep. 30, 2003

Related U.S. Application Data
(62) Division of application No. 09/747,293, filed on Dec. 22, 2000, now abandoned.
(63) Continuation-in-part of application No. PCT/US99/14232, filed on Jun. 23, 1999.
(60) Provisional application No. 60/171,859, filed on Dec. 22, 1999.
(60) Provisional application No. 60/199,469, filed on Apr. 25, 2000.
(60) Provisional application No. 60/090,609, filed on Jun. 23, 1998.

(51) Int. Cl. A61K 38/39

(52) U.S. Cl. 514/12

(57) ABSTRACT

The invention provides methods and compositions that are useful for adhering biological and/or synthetic tissues, sealing fluid and/or gaseous leaks in biological and/or synthetic tissues, and preparing implants useful for delivery of a bioactive molecule such as a drug, for bulking applications, or for tissue prostheses. The present invention also relates to bio-erodable adhesive or occluding compositions and methods of using the same.

This application claims benefit of the filing dates of 60/171,859 filed on Dec. 22, 1999; and 60/199, 469 filed on Apr. 25, 2000.

The disclosures of the following patents and patent applications are incorporated herein by reference: U.S. Pat. Nos. 5,219,895, 5,354,336, and 5,874,537, issued on Jun. 15, 1993, Oct. 11, 1994, and Feb. 23, 1999, respectively; U.S. Ser. No. 09/180,687 based on PCT/US97/08124 filed on May 14, 1997; U.S. Ser. No. 60/090,609 filed on Jun. 23, 1999; PCT/US99/14232 filed on Jun. 23, 1999 designating the U.S.; 60/171,859 filed on Dec. 22, 1999; and 60/199, 469 filed on Apr. 25, 2000.

### BACKGROUND OF THE INVENTION

Fluid and/or gaseous leaks can result from surgeries involving vascular, pulmonary, thoracic, spinal, meningeal, neural, hepatic, lymphatic, digestive, oncological, gynecological and renal tissues. The current standard of care involves the use of hemostats such as thrombin, gelatin and fibrin glue for diffuse bleeding, or the placement of drains until wound resolution for thoracic surgery or lymph node dissections.

A number of surgical sealant compositions also exist but suffer from one or more disadvantages such as handling, biocompatibility, or toxicity. Currently available polymer-based bioadhesives and surgical sealant compositions may also cause unwanted side effects at the tissue sites to which they are applied. Typical side effects include local inflammation, and encapsulation of the material, which results in the formation of fibrous or scar tissue. These side effects can be very detrimental to the health of the patient. For example, neural tissues in both the central and peripheral nervous systems are particularly sensitive to local inflammation, which can result in permanent damage. Ther is therefore a need for tissue sealing methods and compositions that are easy to handle and that do not elicit severe adverse host reactions.

### SUMMARY OF THE INVENTION

The invention provides compositions and methods useful for bonding or sealing tissue, including sealant and adhesive compositions, methods for sealing/adhering fluid and gas leaks, and methods for priming tissues to increase adhesion. The invention also provides methods for controlling the degradation of a sealant or adhesive. Accordingly, the invention provides methods and compositions for reducing the severity of an adverse host reaction to a sealant, which correlates not only with the degree of immunogenicity of the polymeric sealant material but also with the amount of time the material persists at a tissue locus. Useful kits for producing sealants and adhesives are also described.

The present invention depends, in part, on the discovery that the crosslinking of a protein preparation can bond or seal a damaged tissue, and that the bonding of the tissue can be modified by an appropriate selection of proteins and crosslinking agents, by the presence of additives such as surfactants or lipids, and by modifying the pH of the tissue, as described herein. Thus, a leak in a tissue can be repaired using the methods and compositions of the invention, providing a rapid and efficient means to treat a serious or life-threatening condition such as a gas or fluid leak in a tissue.

Useful surgical sealants meet certain performance characteristics. A sealant preferably does not run off of the tissue surface to which it is applied, and it should adhere well to the tissue substrate, be cohesively strong, be compliant, and degrade as the wound heals.

The present invention discloses sealant and adhesive compositions, methods for sealing/adhering fluid and gas leaks, methods for priming tissue to increase adhesion of a sealant or adhesive, and methods for controlling the degradation of the sealant or adhesive. Useful kits for producing sealants and adhesives are also described.

In one embodiment, a composition of the invention for use as a tissue sealant or adhesive comprises a solution of protein and a surfactant preparation, a lipid preparation or a carbohydrate preparation. In preferred embodiments the surfactant, lipid, or carbohydrate are provided at between about 0.1% (w/v), and 10% (w/v) and more preferably between about 0.1% (w/w) and 10% (w/w). In another embodiment, the sealant comprises a protein, surfactant and lipid.

In one embodiment, a method of the invention includes the steps of: (1) providing a lipid, surfactant, and protein in a liquid carrier; (2) providing a crosslinker capable of crosslinking the protein; (3) preparing a sealant by mixing the protein with the crosslinker; and (4) applying the sealant to a tissue, thereby to bond the tissue or seal a fluid or gas leak in the tissue.

In another embodiment, a method for bonding or sealing fluid or gas leaks in tissue includes the steps of: (1) applying to the tissue: (a) a protein preparation, (b) at least one preparation selected from a surfactant preparation and a lipid preparation, and (c) a crosslinker preparation; and (2) permitting the preparation of (1) to form crosslinks, thereby to bond said tissue or seal a fluid or gas leak in said tissue.

In the present invention the protein is preferably albumin, collagen, or globulin and is preferably in solution at a concentration of 3-55% (w/w) of the uncrosslinked solution. The most preferred protein is albumin at a concentration of 25-50% (w/w) of the uncrosslinked solution. The surfactants of the present invention can be either ionic or non-ionic. Preferred surfactants are the alkyl aryl polyetheralcohols, alkanoic acids, perfluoroalkanoic acids, and alkylsulfonic acids. (e.g. tyloxapol, octanoic acid, perfluorooctanoic acid, or sodium lauryl sulfate). The lipids of the present invention may include any natural or synthetic lipid. Preferred lipids are hydrophobically-modified glycerophosphocholines (e.g. dipalmitoylphoshpatidylcholine). The-surfactants and lipids are used to modify such properties as adhesion, and physical and chemical characteristics such as, elongation/tensile moduli, viscosity (rheometry), contact angle and cure time.

A preferred crosslinker is a crosslinker capable of crosslinking a protein. Preferred crosslinkers of the invention are zero-length crosslinkers, in particular carbodiimides. A most preferred carbodiimide is 1-ethyl-3-(3-dimethylaminopropyl) carbodiimide hydrochloride (EDC).

The present invention also describes methods for preparing a tissue to react with a protein-based tissue sealant or adhesive. One embodiment comprises the step of: applying a primer solution at a pH of between about 3.0 to 9.0 to a tissue locus, thereby to prepare said tissue locus for reaction with a protein-based tissue sealant or adhesive. The solution is preferably a buffer with a buffering capacity near the reactive pH of the sealant crosslinker.

In another embodiment, a tissue is prepared to react with a protein-based tissue sealant or adhesive by applying a primer solution containing crosslinker to a tissue locus, thereby to prepare said tissue locus for reaction with a protein-based tissue sealant or adhesive.

In another method, a tissue is prepared to react with a protein-based tissue sealant or adhesive comprising the step of: applying a primer solution containing molecules that promote increased interaction between the sealant and tissue locus, thereby increasing surface area for reaction with a protein-based tissue sealant or adhesive.

The present invention also provides methods for increasing the degradation rate, or reducing the persistence of a polymer-based tissue sealant or adhesive.

One embodiment comprises the step of mixing a polymer degrading agent with a polymer-based tissue sealant or adhesive before applying said polymer-based tissue sealant or adhesive to a tissue locus, thereby increasing the degradation rate of said polymer-based tissue sealant or adhesive at said tissue locus.

In another embodiment for increasing the degradation rate, or reducing the persistence of a polymer-based tissue sealant or adhesive, a polymer degrading agent is applied to a polymer-based tissue sealant or adhesive at a tissue-locus, thereby increasing the degradation rate of said polymer-based tissue sealant or adhesive at said tissue locus.

In particular, the invention is useful to regulate the degradation rate of protein or carbohydrate based bioadhesives or sealants. In a preferred embodiment of the invention, the degradation rate of a polymeric gel is increased in order to increase its degradation in vivo, thereby reducing unwanted side effects associated with prolonged persistence of the gel at a tissue site in a patient.

The current invention also provides a number of useful kits based on preferred compositions and methods:

In one embodiment, a kit for producing a protein-based tissue adhesive or sealant comprises: (1) a tissue primer, (2) a protein preparation, (3) at least one preparation selected from a surfactant preparation and a lipid preparation (4) a cross-linker preparation, and (5) a preparation of protein degrading agent.

In an alternative embodiment, a kit for producing a protein-based tissue adhesive or sealant comprises: (1) a protein preparation, (2) at least one preparation selected from a surfactant preparation or a lipid preparation, and (3) a cross-linker preparation, and that may further comprise at least one preparation selected from: (a) a tissue primer, and (b) a preparation of protein degrading agent.

In another embodiment, a kit for producing a protein-based tissue adhesive or sealant comprising: (1) a protein preparation, (2) a cross-linker preparation.

In a further embodiment, a kit for producing a protein-based tissue adhesive or sealant comprising: (1) a protein preparation, (2) a preparation of protein degrading activity, and (3) a cross-linker preparation.

### DETAILED DESCRIPTION OF THE INVENTION

The present invention provides sealant and adhesive compositions, methods for sealing or adhering fluid and gas leaks, methods for priming tissue to increase adhesion of a sealant or adhesive, and methods for controlling the degradation of the sealant or adhesive. Useful kits for producing sealants and adhesives are also described.

The compositions, methods, and kits, are useful sealants and adhesives for adhering and/or sealing fluid and/or gaseous leaks in biological and/or synthetic tissues. Such methods are particularly useful for surgical procedures such as vascular, cardiovascular, pulmonary, renal, hepatic, general, digestive, neural, and spinal procedures.

As used herein, “tissue” means a biological tissue or a synthetic tissue. A biological tissue includes connective tissues, endothelial tissues, nervous tissues, muscle tissue and organs. Preferred biological tissues are selected from the group consisting of bone, skin, cartilage, spleen, muscle, lymphatic, renal, hepatic, blood vessels, lung, dura, bowel and digestive tissue. A synthetic tissue includes synthetic tissue made from biological material or synthetic tissue made from synthetic material, and may further include biological materials such as cells, or bioactive molecules. Examples of synthetic tissue include expanded poly(tetraflouroethylene) (PTFE), polyester, or other synthetic materials used to manufacture an implant such as a prosthesis.

Composition of the Sealant

According to preferred embodiments of the invention, a sealant or implant is based on a protein component. The protein component may comprise any natural protein, peptide or polypeptide such as collagen, albumin, globulin, fibrin, elastin, histone, laminin, protamine, or serum fraction protein, or any combination thereof. The protein component may also comprise synthetic proteins, peptides, or polypeptides or any combination thereof. In this invention, a synthetic protein, peptide or polypeptide is defined as any protein, peptide or polypeptide that has been chemically or recombinantly modified or produced.

In alternative embodiments of the invention, a sealant or implant is based on a carbohydrate component. Preferred carbohydrates include alginates and pectins. In a further embodiment a sealant or implant is based on a mixture of protein and carbohydrate. Carbohydrates are useful to modify the viscosity and/or elasticity of a protein-based sealant. For example, the addition of pectin or alginate to an albumin-based sealant, prior to cross-linking, increases the viscosity of the reaction mixture and results in increased elasticity of the final gel.

Proteins

in accordance with the present invention, a choice of protein(s) to use and the concentration needed depend on well-defined factors described herein. Factors include how the final product will be used, target tissue, desired degradation rate, and physical/chemical properties. For example, a sealant must meet certain specifications determined by the specific needs of the substrate. The sealant will have to withstand the normal pressures the tissue is under (e.g. 120 mm Hg for blood vessels) as well as conform to the natural transitions at the tissue site (e.g. have a high elastic modulus in the case of lung tissue applications). Assays useful for optimizing the compositions and methods of the invention are disclosed herein, and exemplary protein-based compositions and tissue specific applications are provided in the Examples.

The concentration of protein in the crosslinked sealant or adhesive ranges from about 3 to 55% (w/w). The actual concentration of protein used is dependent on variables such as protein solubility, desired physical properties following crosslinking (e.g. tensile strength, elasticity, hardness), as described herein. The concentration will also have an affect on how long a sealant or adhesive persists in vivo.

Preferred proteins of this invention include albumin, collagen, gelatin, protamine, histones and globulins. The concentration of albumin is preferably between 10 and 55% w/w, and more preferably between 25 and 45% and most preferably between 30 and 40% w/w. The concentration of collagen is preferably between 3 and 12% w/w, and more preferably between 5 and 10%. The concentration of globulin is preferably between 10 and 35% w/w, and more preferably between 15 and 30%, and most preferably between 20 and 25% w/w.

In any one of the above embodiments, the sealant monomer may be in solution with or covalently bound to a molecule selected from the group consisting of nucleotides, peptides, synthetic polymers, carbohydrates (e.g., alginates), polysaccharides (e.g., glycosaminoglycans, dextrans, hyaluronic acid, chondroitin sulfate, heparan sulfates), polyethers (e.g., polyethylene glycol, polypropylene glycol, polybutylene glycol), polyesters (e.g., polylactic acid, polyglycolic acid, polysalicylic acid), aliphatic, alicyclic, aromatic, perfluorinated or non-perfluorinated, and other derivatizing agents. In addition, the monomer preparation may contain a chlorinated, fluorinated, brominated or iodinated derivative.

Albumin

In some embodiments, albumin-based sealants are preferred. Preferably, the albumin is of mammalian origin, but other sources of albumin also may be employed. It is believed that most albumins are readily cross-linked according to the methods of the invention. However, an albumin with low immunogenicity is preferred for in vivo applications. Accordingly, for uses in humans, it is preferred that the albumin is human albumin. Bovine serum albumin (BSA) may also be used in humans, and is more readily available. Alternatively, the albumin may be recombinant albumin, isolated from cells expressing a recombinant albumin gene, using methods known in the art. When produced recombinantly for use in humans, the albumin gene is preferably a human or bovine gene. However, other species or biosynthetic variants may be used. Major fragments of albumin, comprising at least 100 residues of an albumin sequence, whether produced by partial proteolysis or by recombinant means, may also be used instead of intact albumin. Alternatively, useful fragments may contain at least 50 residues, and more preferably at least 75 residues of an albumin sequence. Finally, mixtures of different forms of albumin (e.g., human, bovine, recombinant, fragmented), and plasma fractions rich in albumin may also be employed.

Albumin may be purified directly from tissues or cells, using methods well known in the art (see, e.g., Cohn et al. (1946) J. Amer. Chem. Soc. 68:459; Cohn et al. (1947) J. Amer. Chem. Soc. 69:1753; Chen (1967) J. Biol. Chem. 242:173). Alternatively, albumin may be purchased from a commercial supplier. For example, albumin preparations from various mammalian and avian species may be purchased from Sigma Chemical Company (St. Louis, Mo.) in the form of solutions or lyophilized powders. A preferred commercial supplier of albumin is Intergen (Purchase, N.Y.).

In preferred embodiments, albumin is provided as an aqueous solution of 10-55%, preferably 25-45%, and most preferably about 30%-40% albumin by weight. As explained more fully below, lower concentrations of albumin may be employed when viscosity-enhancing agents are added. In some embodiments, the solution is preferably substantially purified to remove contaminants such as immunogens that would disrupt or interfere with the bioadhesive or sealant properties of the cross-linked albumin. On the other hand, the presence of many other proteins, such as collagen, elastin, laminin, fibrin, and thrombin, can be tolerated.

Alternatively, albumin may be provided as a dry powder. In such embodiments, the dry albumin is solubilized at the site of administration. Thus, body fluids (such as blood) present at the site of administration may be sufficient to solubilize the protein. Alternatively, additional fluids may be provided along with the dry albumin. The cross-linker may also be provided as a dry powder that is solubilized at the site of administration. In a preferred embodiment, the dry protein and cross-linker are mixed prior to administration. In a most preferred embodiment, a wetting reagent is added to the protein and cross-linker mixture in order to increase fluid absorbance. Preferably, the wetting reagent absorbs water from the available body fluids and speeds up solubilization of the protein and cross-linker.

Albumin may be modified or derivatized to increase viscosity. For example albumin viscosity may be increased by adding in solution or covalently attaching relatively large (10-100 kD), substantially linear molecules such as polysaccharides (e.g., glycosaminoglycans, dextrans, hyaluronic acid, chondroitin sulfate, heparan sulfates), polyethers (e.g., polyethylene glycol, polypropylene glycol, polybutylene glycol), polyesters (e.g., polylactic acid, polyglycolic acid, polysalicylic acid), and aliphatic, alicyclic or aromatic acylating or sulfonating agents. Preferred acylating agents including aliphatic, alicyclic and aromatic anhydrides or acid halides, particularly acid anhydrides of dicarboxylic acids, Non-limiting examples of these include glutaric anhydride, succinic anhydride, lauric anhydride, diglycolic anhydride, methacrylic anhydride, phthalic anhydride, succinyl chloride, glutaryl chloride, and lauroyl chloride. The acylating agents may also include various substituents and secondary functionalities such as aliphatic, alicyclic, aromatic and halogen substituents, as well as amino, carboxy, keto, ester, epoxy, and cyano functionalities, and combinations thereof. Similarly, preferred sulfonating agents useful in the invention include aliphatic, alicyclic and aromatic sulfonic acids and sulfonyl halides, which may also include various substituents and secondary functionalities as described above.

Albumin also may be modified or derivatized to increase its hydrophobicity in order to promote interactions with hydrophobic tissues or prosthetic materials. Specifically, albumin may be derivatized with branches or straight chain alkyl, alkenyl, or aromatic reagents, including long chain alkyl or alkenyl and alkyl aldehydes or carboxylic acids such as octyl or dodecyl aldehyde or carboxylic acid.

Finally, in order to increase its hydrophobicity and its ability to interact with fluorine-containing prosthetic materials (e.g., PTFE-containing materials), albumin or modified albumin may be halogenated, preferably fluorinated, by standard methods well known in the art. For example, albumin may be derivatized with polyfluoro dicarboxylic acid anhydrides (e.g., hexafluoro glutaric anhydride), polyfluoro aklyl ethers (e.g., perfluoroalkyl glycidyl ethers), or other halogen containing reagents.

Alternatively, a recombinant albumin may be produced by standard techniques of site-directed mutagenesis in which one or more amino acid residues are inserted, deleted or substituted to increase the viscosity of the albumin, to alter the hydrophobicity of the protein, to provide more side chains for derivatization, or to provide more free carboxyl or amine groups for the cross-linking reaction. As a general matter, under the conditions employed, albumin contains an adequate (and roughly equal) number of free carboxyl and amine groups for cross-linking. Therefore, it is anticipated that modifications of the albumin sequence will be most useful for increasing the viscosity of the protein by replacing small or hydrophilic residues (e.g., glycine, alanine) with larger and/or more hydrophobic and/or charged residues which can participate in non-covalent intermolecular bonds through charge-charge or hydrophobic interactions. Alternatively, however, one may produce two forms of modified albumin differing substantially in their free carboxyl and amine contents.

Carbohydrates

Preferred carbohydrates are natural or synthetic poly- and oligo-saccharides. Preferred carbohydrates include amylose, amylopectin, alginate, agarose, cellulose, carboxymethyl cellulose, carboxymethylamylose, chitin, chitosan, pectin, and dextran.

Crosslinkers

According to the invention a polymer is crosslinked to form a sealant or adhesive. The crosslinking agent can be any crosslinker capable of crosslinking protein, including crosslinkers known in the art as well as any new crosslinkers discovered in the future. Crosslinkers useful in the invention can be divided into two general classes. The first class includes crosslinkers that activate functional groups on proteins to react with each other (e.g. carbodiimides, oxidants, deprotectants). The second class includes crosslinkers containing functional groups that react with other functional groups on the proteins to form molecular bonds (e.g. multielectrophilic PEG, multialdehyde).

Crosslinkers include zero-length crosslinkers, homobifunctional crosslinkers, heterobifunctional crosslinkers, and multifunctional crosslinkers, or any other crosslinker that produce any combination of ionic, covalent, intermolecular, or intramolecular bonds. Non-limiting examples of crosslinkers include carbodiimides, isoxazolium salts, carbonyidiimidazole, electrophilic crosslinkers such as di- and multi-aldehydes, di- and multi succinimidyl esters, sulfhydryl oxidation. In addition, the crosslinker may be covalently bound to the protein or free as a secondary molecule.

In some embodiments, a crosslinker that forms reversible crosslinks may be used to promote degradation of the sealant in the body. Thus, a crosslinker may contain or form an unstable bond including, for example, a disulfide, lactone, lactam, ester, thioester, acetal, ketal, thioacetal, thioketal, or imidoamide.

The preferred crosslinker of this invention is a carbodiimide, in particular the water soluble 1-ethyl-3-(3-dimethylaminopropyl carbodiimide) hydrochloride (EDC-HCl).

Carbodiimides

Carbodiimides are cross-linking reagents having the general formula:
R₁—N═C═N—R₂.

In general, carbodiimides react with carboxyl groups to form a reactive intermediate. This reactive intermediate subsequently reacts with a nucleophile (e.g. amines, hydroxyl, sulfhydryl, etc.) to form a bond (e.g. amide, ester, thioester, etc.) and a urea based byproduct. The chemistry is outlined in the following general reaction:
R₃—COOH+R₁—N═C═N—R₂→R₃—COOC—N—R₁(═N—R₂)
R₃—COOC—N—R₁(═N—R₂)+R₃—NH2→R₃—CONH—R₄+R₁—NH—CO—NH—R₂

Carbodiimides are reactive over a wide pH range (1-9.5). At alkaline pH (>8) the reaction is slow, but as the pH decreases the reaction rate increases. However, at low pH hydrolysis of the carbodiimide competes with the formation of crosslinks. Thus, the reaction is most efficient in a pH range of 5-7. The pH-sensitivity of the reaction permits control over the speed of the reaction and the density of crosslinking. The reaction speed can be measured using a cure-time assay, and crosslink density can be measured indirectly using tensile strength as described in the analytical methods section.

According to the invention R1 and R2 may be the same or different and may be any chemical group that does not react with the carbodiimide. R₁ and R₂ are typically selected from the group consisting of any straight or branched chain, saturated or unsaturated, alkyl, alkenyl, aryl, aralkyl, or aralkenyl group, or variants thereof with halogen, tertiary amino, quaternary amino, ester, keto, polyalkylene oxide or other substituents. In addition, one or both of R₁ and R₂ may include an additional carbodiimide group, such that the cross-linker is a polycarbodiimide.

Preferably, the carbodiimides employed are water-soluble. However, a suspension of water insoluble carbodiimide may also be useful for cross-linking if sufficiently dispersed in the cross-linking reaction. By appropriate choice of R groups, the solubility and reactivity of the carbodiimide may be varied. In addition, the choice of R groups will affect the immunogenicity and toxicity of the cross-linker, as well as its ability to interact with the biopolymer molecules.

The carbodiimide may be provided as a solution or suspension. However, since carbodiimides are subject to hydrolysis they are usually provided in dry form, such as a powder. The dry carbodiimide is solubilized or suspended before it is mixed with the device or administered to the tissue. It may also be solubilized by body fluids present at the site of administration, as may be the case in a sponge-based sealant, or if being used as a primer for tissue crosslinking activation.

In another embodiment, the carbodiimide may be provided in a solution of an inert material. Examples of inert materials include tetrahydrofuran, glycerol, triglycerides, poly(vinyl alcohol), polyalkylene oxides (polyethylene glycol, polypropylene glycol), non-ionic surfactants, including PEG-based surfactants such as pluronic polymers and other inert polymers and soybean oil or tyloxapol.

Examples of useful carbodiimides include 1-(3-dimethylaminopropyl)-3-ethylcarbodiimide; 1,3-di-p-tolylcarbodiimide; 1,3-diisopropylcarbodiimide; 1,3-dicyclohexylcarbodiimide; 1-cyclohexyl-3-(2-morpholinoethyl) carbodiimide metho-p-toluenesulfonate; polycarbodiimide; 1-tert-butyl-3-ethylcarbodiimide; 1,3-dicyclohexylcarbodiimide; 1,3-bis(trimethylsilyl)carbodiimide; 1,3-di-tert-butylcarbodiimide; 1-(3-dimethylaminopropyl)-3-ethylcarbodiimide methiodide; and 1-(3-dimethylaminopropyl)-3-ethylcarbodiimide, all available from the Aldrich Chemical Company, Milwaukee, Wis.

The preferred carbodiimide of this invention is the water soluble 1-ethyl-3-(3-dimethylaminopropyl) carbodiimide hydrochloride (EDC-HCl).

A typical sealant or adhesive is prepared by mixing a protein component with a crosslinking component. The reaction conditions for crosslinking a protein with a carbodiimide depend on the desired properties of the crosslinking reaction and the final gel. Herein the term “gel” refers to the crosslinked sealant or adhesive. Desired properties include time to gellation, time to complete cure and crosslink density. Reaction conditions include concentration of carbodiimide and protein, pH of the mixed protein/crosslinker solutions, and the ratio of carbodiimide solution volume to protein solution volume.

Reaction Conditions Using Carbodiimides

The amount of carbodiimide needed to crosslink a protein to form a sealant or adhesive depends on well defined variables and may be optimized as described herein. Optimization may be based on the measurement of cure time, and the strength of a gel over time. In practice, estimating the amount of carbodiimide to be employed in a biopolymer system can be done by two methods. Following this the concentration and pH of the crosslinker and sealant solution can be adjusted to obtain the required gel time and gel strength.

In the first method one equivalent of carbodiimide is used per equivalent of limiting functional group (e.g. carboxylic acid or nucleophilic groups (amine, sulfhydryl) of the protein at a particular pH (usually 5-7). Depending on the gellation time from this experiment, the amount of carbodiimide may be adjusted and optimized for a particular application.

Following is an example demonstrating this method. The example is for crosslinking 1 g of a bovine serum albumin (BSA) solution at a concentration of 40% w/w (represents 0.4 g BSA), where the crosslinker is EDC-HCl. BSA has a molecular weight of approximately 67,000 and contains 101 moles carboxylic acid/mole BSA (carboxylic acids being part of aspartic and glutamic acid residues and at the C-terminus) and 60 moles amine/mole BSA (amines being part of lysines and at the N-terminus), therefore the amine is the limiting functional group. The following calculation determines the amount of EDC-HCl to try first:

$$0.4 g BSA \times \frac{1 mole BSA}{67000 g BSA} \times \frac{60 mole amine}{mole BSA} \times \frac{191.7 g EDC \text{-} HCl}{M ole EDC \text{-} HCl} = 0.069 g$$

In the second method a weight ratio (carbodiimide:protein) of 1:20 is used as a starting point. This method would be useful if the exact chemistry of the starting protein is unknown and limiting functional groups cannot readily be determined.

It should be recognized that these two methods are only a means of arriving at a starting point for determining the final amount of carbodiimide needed. Other variables also effecting the amount of carbodiimide needed include the pH of the protein solution, effect of additives, ratio of crosslinker solution to protein solution and viscosity/effectiveness of mixing. Accordingly, it may be necessary to adjust the amount of carbodiimide.

The above examples represent two methods for determining the ratio of carbodiimide to protein, however it is recognized that other methods for determining the ratio exist and are known to those skilled in the art.

In the present invention useful ranges of weight carbodiimide:weight protein include 1:80 to 1:1, with a more preferred range of 1:40 to 1:5. However, each individual polymer system may be different and optimal ratios may be determined as described herein. For example, a preferred range for albumin-based sealants is 1:10 to 1:20, and most preferably about 1:14 to 1:16.

The ratio of crosslinker solution volume (CSV) to protein solution volume (PSV) will affect the overall strength of the gel. For example, compare mixing a one to one ratio of 40% albumin to 2% EDC-HCl with a five to one ratio of the same solutions. The 1:1 solution will result in a weaker, more easily deformed gel with only 20% w/w crosslinked albumin while the 5:1 will result in a stronger, more robust gel with 32% w/w crosslinked albumin. This dependency on the ratio of PSV:CSV on the final properties of the gel allow for some control over the final properties of the gel.

The concentration of the carbodiimide solution depends on the mixing and delivery system and the effect it has on the final device's properties.

Crosslinking Proteins Using Carbodiimide Mediated Reactive Esters

In another embodiment a secondary crosslinking molecule may be added with the carbodiimide to affect the rate, extent, and operational range of a crosslinking reaction. For example, in the case of carbodiimide crosslinking the reactive intermediate is short lived and works best in the pH range of 5-7. In some circumstances it may be more advantageous to crosslink at a higher pH or to have a reactive intermediate with a longer half life, especially if the concentration of reactive nucleophile (amine, sulfhydryl, hydroxyl) is limited. In one embodiment of the invention the additive is a molecule that will react in conjunction with a carbodiimide to form another reactive group. In a preferred embodiment the additive forms a succinimidyl, nitrophenol, or maleimide reactive ester. The most preferred additives of the present invention are N-hydroxysuccinimide (NHS) and N-hydroxysulfosuccinimide (sulfoNHS).

The concentration of NHS and suffoNHS are preferably between 0.1 and 50% (w/w), more preferably between 1 and 25% (w/w) and most preferably between 5 and 15% (w/w).

Lipids

According to the invention a lipid may be provided with the sealant or adhesive. Natural lipids are water-insoluble, oily or greasy organic substances that are extractable from cells and tissues by nonpolar solvents such as chloroform or ether. Lipids also include synthetic lipids and synthetic variants of natural lipids. In one embodiment, a lipid may be added to a sealant or adhesive to increase wetting into a hydrophobic surface; in another mbodiment the lipid may be added to increase the elasticity of the sealant or adhesive. For example, DPPC increases the elasticity of an albumin gel cross-linked with carbodiimide. Since lipids are insoluble in aqueous solutions, it may need to be used in conjunction with a surfactant. Exemplary lipids include phospholipids such as phosphoglycerides or sphingomyelin, glycolipids, and sterols. Preferred lipids include phosphoglycerides such as phosphatidyl cholines, phosphatidyl serines, phosphatidyl ethanolamines, phosphatidyl inositols, and diphosphatidyl glycerol. For applications involving lung tissue, preferred lipids, if present, include phosphatidyl cholines. One particularly useful set of lipids is the set of hydrophobically substituted glycero-phosphocholines with a structure of R₁—C(O)—O—CH₂—(R₂—C(O)—O)CH₂—CH₂—OPO₂(CH₂)₂—N(CH₃)₃, where R₁ and R₂ are typically saturated and/or unsaturated alkyl groups ranging in size from C4 to C22 and may either be the same or different. For example, a useful device that interacts well with lung tissue is composed of dipalmitoylphosphotidylcholine (DPPC, R₁ and R₂ are C16) dispersed into an albumin solution containing the non-ionic surfactant tyloxapol.

Other insoluble modifiers that may also be used include but are not limited to hydrophobically modified phosphatidic acid (e.g. dipalmitoylphospatidic acid, dilaurylphosphatidic acid), phosphotidylethanolamine, phosphotidylinositol, alkyl-glucopyranosides, long chain fatty alcohols, and bile acids.

The type and concentration of lipid is dependent on the application. The preferred concentration of lipid is 0.1 to 10%, more preferably 2-8%, and most preferably 3-7%.

Surfactants

According to the invention a surfactant may be provided with the sealant or adhesive in order to effect some physical or chemical property of the sealant or bioadhesive or some other component. Surfactants of this invention are compounds that lower the surface tension of water. Surfactant molecules preferably contain a hydrophobic end of one or more hydrocarbon chains and a hydrophilic end.

In one embodiment of the invention, the surfactant is ionic. Ionic surfactants are charged. Ionic surfactants of this invention include fatty acids (linear and branched alkanoic acids), linear and branched alkylbenzenesulfonates, linear and branched alkanesulfonates, alkylamines, quaternary aminoalkanes, perfluoroalkanoic acids, perfluoroalkanesulfonates. Non-limiting examples of these include octanoic acid, dodecanoic acid, palmitic acid, sodium lauryl sulfate, perfluorooctanoic acid, and perfluorosuberic acid, all available from Sigma-Aldrich.

The preferred ionic surfactants are octanoic acid, palmitic acid, perfluorooctanoic acid, and sodium lauryl sulfate.

In another embodiment of the invention the surfactant is non-ionic. Non-ionic surfactants contain a hydrophobic region with an uncharged hydrophilic region to impart aqueous solubility. Non-ionic surfactants of this invention include alkyl aryl polyether alcohols, and alkyl- or perfluoroalkyl-polyoxyethylene ethers, polyoxyethylene esters, polyoxyethylene sorbitan. Non-limiting examples of these include tyloxapol, Brij 58, Zonyl 100, all available from Sigma-Aldrich.

According to the invention a surfactant should be between 0.05 and 10%. However, the choice of surfactant and the concentration needed depend on the intended use. A surfactant may be chosen as a function of the specific sealant composition or tissue application as described herein.

In one aspect of the invention the surfactant is added to promote wetting of the sealant or adhesive into the tissue. Wetting is defined as the ability of a liquid to interact with a solid surface, and is analogous to solvents where like dissolves like. The affinity or interaction of a liquid for a solid can be measured indirectly using contact angle. In general a lower contact angle indicates a higher degree of interaction. In the case of sealants and adhesives of this invention, it is advantageous to have high interaction between the target tissue and the sealant/adhesive, since good wetting and spreading increases the contact area providing more opportunity for bonding. According to the invention a surfactant may be included with a sealant composition to increase the sealant's ability to interact with a target tissue by matching the chemical characteristics (e.g. hydrophobicity) of the tissue to promote wetting of the sealant or adhesive into the tissue substrate. For example, perfluorooctanoic acid can be added to a sealant solution to allow for better wetting into expanded PTFE. In another example octanoic acid or sodium lauryl sulfate is added to a sealant to allow better wetting into the hydrophobic lung surface.

In another aspect of the invention the surfactant is added to disperse an insoluble lipid component. For example tyloxapol may be included in a sealant to disperse an insoluble lipid.

In yet another aspect the surfactant is added to increase viscosity. For example, if the tyloxapol concentration of an albumin solution is increased the viscosity increases. This viscosity increase is due to a denaturation of the protein. It should be recognized that other means of denaturation (e.g. sodium lauryl sulfate, urea, heat, etc.) would also increase viscosity.

Degrading Agents

Methods of Controlling the Degradation of Polymer Based Tissue Sealants or Adhesive

The current invention also provides methods related to regulating the rate of degradation of polymeric gels used in medical applications. The invention is particularly useful to increase the rate of degradation of polymer-based bioadhesives or sealants, thereby increasing their rate of degradation in vivo. Faster degradation of these sealants or bioadhesives prevents or reduces unwanted adverse reactions associated with their presence (in the form of a bioadhesive or sealant) at a tissue site in the body of a patient.

According to the invention, one method for increasing the degradation rate, or reducing the persistence of a polymer-based tissue sealant or adhesive comprising the step of mixing a polymer degrading agent with a polymer-based tissue sealant or adhesive before applying said polymer-based tissue sealant or adhesive to a tissue locus, thereby increasing the degradation rate of said polymer-based tissue sealant or adhesive at said tissue locus.

A method for increasing the degradation rate, or reducing the persistence of a polymer-based tissue sealant or adhesive comprising the step of applying a polymer degrading agent to a polymer-based tissue sealant or adhesive at a tissue locus, thereby increasing the degradation rate of said polymer-based tissue sealant or adhesive at said tissue locus.

Polymeric gels used for medical applications are typically biocompatible. However, most biocompatible polymeric gels are nonetheless antigenic (even if only minimally so) and do elicit a host immune response, especially if they are present in large amounts and have a prolonged persistence in the patient. A polymer may also be biocompatible, but the host may not have the physiology to break down the polymer. For example, plant derived polymers (e.g. polysaccharides) may not be readily broken down by metabolic mechanisms present in humans.

Therefore, the prolonged presence of useful polymer-based bioadhesives, sealants, and implants may cause unwanted side effects at the tissue sites they are applied to. Typical side effects include local inflammation and encapsulation of the polymeric gel that results in the formation of scar tissue. These side effects can be very detrimental to the health of the patient. For example, neural tissue (nerves and central nervous system) is particularly sensitive to local inflammation. It will be apparent to one of ordinary skill in the art that the severity of an adverse host reaction at a tissue locus correlates not only with the level of bio-incompatibility of the polymeric gel but also with the amount of time the gel persists at the tissue locus.

The adverse host reaction to the presence of a polymeric gel is prevented or reduced by increasing the degradation rate of the gel. In general, faster degradation of a bioadhesive, sealant, or implant results in less host reaction. In a preferred embodiment, the degradation rate of the gel is optimized to permit the gel to persist for a time sufficient to perform its function (e.g. binding, sealing), but no longer than necessary. For example, a sealant should degrade at the rate of healing. The optimal rate of gel degradation is a function of the wound healing rate. Methods for measuring and optimizing the rate of gel degradation are described herein, and exemplified in Example 2.

Methods of the invention are particularly useful to increase the degradation rates of polymeric gels that degrade slowly in vivo. For example, the invention is useful to enhance the degradation of albumin-based gels, which are typically degraded very slowly in vivo.

According to the invention, methods for regulating the degradation of a polymeric gel comprise providing an additive that alters the gel's degradation rate. In preferred embodiments, the invention comprises 1) providing a degradation factor (for example a degradation enzyme), 2) providing a stimulatory factor that stimulates or enhances a natural tissue-associated gel degradation activity or 3) providing any combination of additives 1 and 2. In another preferred embodiment the invention comprises providing an inhibitory factor that inhibits or reduces a natural tissue associated gel degradation activity.

The type of additive that is provided is a function of the type of polymer being degraded. Polymers contemplated by the invention comprise those useful as sealants or adhesives. Typical sealants are formed by mixing a structural (polymer) component with a cross-linking or polymerizing agent under conditions to promote cross-linking or polymerization of the structural component to generate a gel. Polymers used as sealants or adhesives are generally cross-linked or polymerized in situ at the site of a wound or other tissue injury being treated. In preferred embodiments of the invention, the sealant or adhesive is protein-based. However, the invention also contemplates regulating the degradation of gels based on carbohydrates, nucleic acids, synthetic polymers, and any combination of above mentioned polymers.

In a preferred embodiment, a degradation factor or a stimulatory factor is added to either the structural component of the sealant or bioadhesive, the cross-linking or polymerizing agent, or both, before the sealant or bioadhesive is formed via cross-linking or polymerization. The degradation or stimulatory factor is thereby incorporated throughout the sealant or bioadhesive. The degradation or stimulatory factor may be cross-linked to the structural components of the sealant or adhesive, or may remain in solution. According to the invention, the additive is preferably provided in solution, but optionally in suspension or in dry powder form. In an alternative embodiment, a degradation or stimulatory factor is added to the formed gel after cross-linking or polymerization. The degradation or stimulatory factor is applied to the surface of the gel. According to the invention, the additive is preferably applied as a solution, but optionally as a suspension or a dry powder.

One of ordinary skill in the art will appreciate that the amount of degradation agent or stimulatory factor provided to a sealant or bioadhesive is a function of several well-defined factors described herein, including the desired degradation rate, the type of polymer used, the activity of the added material, the crosslinking density, and antigenicity of a sealant, adhesive, or implant.

In preferred embodiments of the invention, the additive is provided in an amount sufficient to result in degradation of the sealant or bioadhesive when it is no longer needed. An adhesive or a sealant is typically provided to temporarily bond two tissues together, or seal a fluid or gas leak in a tissue. The adhesive or sealant properties preferably persist while the wound heals. According to the invention, once the wound has healed sufficiently to be structurally stable in the absence of the adhesive or sealant, then the sealant or bioadhesive is degraded. In most embodiments of the invention, the gel is degraded in less than about 100 days. In preferred embodiments, the gel is degraded in less than about 50 days, and most preferably in less than about 30 days.

Accordingly, in one embodiment of the invention, an additive is provided in an amount sufficient to increase the sealant or bioadhesive degradation. As used herein the term “degradation” means the breaking of molecular bonds (covalent, ionic, hydrogen) within the biopolymer or those formed by crosslinking or a combination of both, resulting in the breakdown of the structural integrity of the crosslinked sealant or bioadhesive. Preferably, degradation results in disappearance of the sealant, adhesive, or implant.

In the context of sealant or tissue degradation at a tissue site in a patient, the sealant or bioadhesive degradation products may be soluble and removed from the tissue site as it is degraded. Alternatively, the degradation products may be insoluble sealant or bioadhesive fragments, or may form a precipitate at the tissue site as the sealant or bioadhesive is degraded. In preferred embodiments, these fragments or precipitated degradation products are removed by natural processes such as phagocytosis.

In a preferred embodiment, degradation results from the cleavage of covalent bonds in the sealant or bioadhesive structure. In a sealant or bioadhesive that is formed by chemically cross-linking multiple gel subunits (e.g. proteins or carbohydrates) to form a cross-linked product, degradation can result from cleavage of covalent bonds within the subunits, or from cleavage of the bonds formed by the chemical cross-linkers, or from a combination of both.

Typically, there are many possible cleavage sites within a sealant or bioadhesive contemplated by the invention. For example, a protein-based sealant can theoretically be degraded by cleavage at any one or more peptide bonds within the protein amino acid sequence. However, in practice, only a subset of these sites are cleaved as the sealant is degraded. The cleavage sites are determined by the structure of the sealant and sealant subunits, and also by the substrate specificity of the cleavage agent or factor (for example, most proteases have preferred cleavage sites).

At a gross structural level, sealant degradation can be observed after cleavage of only a subset of the bonds holding the sealant together. From the point of view of the gross structure of the sealant, degradation can occur in different ways. The outer layer or surface of the sealant may be degraded first thereby exposing inner layers or surfaces of the sealant to further degradation. Accordingly, the sealant progressively shrinks in size over time. Alternatively, bonds are cleaved throughout the sealant thereby progressively destabilizing the entire structure. Accordingly, the sealant initially weakens and eventually breaks up into small fragments. These fragments may then be further degraded. The type of degradation depends on a number of factors, including whether the degradation agent or factor is applied to the surface of the sealant or is present throughout the sealant; whether the agent is only active on the surface, or is active throughout the sealant; and the structure of the sealant.

In preferred embodiments of the invention the host response is reduced by reducing the presence of the sealant, resulting in less inflammation, hemorrhaging, encapsulation and formation of scar tissue.

The amount of additive required is a function of the activity of the additive, the structure of the sealant, and the tissue application. Typical molar ratios of the structural components to the additive of the sealant are preferably from 1:10 and 20,000:1, more preferably from 100:1 to 5000:1, and most preferably from 1000:1 and 2500:1.

In the case of protein based sealants the amount of protease to add will be a function of the concentration of the protein in the sealant, the specific activity of the protease sample, the amount of available cleavage sites on the protein, the specific turnover of the enzyme, and crosslink density of the final gel.

The specific activity of the protease is defined as the units per milligram of solid protein. The unit definition will vary depending on the enzyme-substrate combination used; the preferred definition is the one used in the US Pharmacopoeia guidelines. Once the specific activity has been determined, it will be necessary to determine the potential cleavage sites available on the protein. Proteases typically cleave around one or more specific amino acid residues and the potential sites will approximate the amount of those residues present in the sequence of the protein.

To calculate the amount of enzyme to add, the active sites will be calculated per ml of formulation used. It is not necessary to cleave all of the potential sites, and the amount of enzyme should cleave from 1 to 50% of the potential sites, depending on the desired time of degradation. The following formula can be used to determine the amount of enzyme: Moles of cleavage sites (MCS): (Moles of protein/ml)×(Moles of cleavage sites/Mol of protein). Moles used in crosslinking (MUC): grams of crosslinker/molecular weight of crosslinker (assume 100% efficacy). Units of protease: (MCS-MUC)X % of potential sites (Where X=1-50).

The above calculation is affected by the following. Catalytic constants are calculated based on small substrate analogs, so steric hindrance will not affect its activity. When the substrate is many sites on a protein some of them will not be available, resulting in a decrease in the substrate concentration.

The constants are also calculated in solution. In the case of sealants the enzyme is immobilized, which will greatly reduce its turnover number since it can only catalyze the cleavage of neighboring peptide bonds, resulting in a loss of catalytic efficacy.

Its not necessary to cleave all the theoretical sites to get enough degradation since the in vivo processes will also be contributing to the degradation of the gel.

If the crosslink density of the protein is increased, then there should be a proportional increase in the amount of enzyme added to counter the increased possibility of losing proteolytic activity due to inhibited enzyme.

It is contemplated, however, that the optimal amounts of additive provided, and the optimal modes of administering the additive may be determined by routine experimentation well within the level of skill in the art. Example 2 describes experiments useful to optimize the amount of additive that is provided to a sealant or adhesive of the invention.

In embodiments where the sealant is particularly antigenic, or if the tissue site of application is particularly sensitive, the degradation of the gel is optimized to be rapidly degraded when it is no longer needed. In other embodiments, where the gel is very biocompatible, or where the tissue is tolerant of a host immune response, the degradation rate of the gel does not need to be as carefully optimized. However, an additive is preferably provided to ensure that the gel is eventually degraded.

In most preferred embodiments of the invention, the degradation rate of the gel is non-linear. Preferably, little or no degradation occurs when the gel is first applied to the tissue, and the degradation rate progressively increases over time. An additive may be provided in an inactive form, and subsequently activated to degrade the gel. For example, the additive may be an inactive form of a protease that is processed (either self-processed, or by a separate processing activity provided along with the protease) to produce an active protease. The amount of time before gel degradation occurs is controlled by controlling the processing rate of the protease.

Methods of the invention are preferably used to regulate the degradation of sealants or bioadhesives based on the following structural elements: proteins, carbohydrates, and nucleotides including naturally occurring and synthetic variants, and other synthetic polymers, or any combination thereof.

Proteases

According to one embodiment of the invention, including a protease in the polymer reaction mixture regulates the degradation of protein-based polymers. Naturally occurring proteases are preferred degrading agents for protein-based polymers. However, modified proteases are also contemplated by the invention. Modified proteases include chemically derivatized proteases and recombinantly modified proteases. In one embodiment, a modified protease with altered proteolytic activity [e.g. increased substrate specificity and catalytic activity] is used to obtain optimal specificity and degradation rate.

Naturally occurring proteases contemplated by the invention include the following types of proteases:
serine proteases, including but not limited to chymotrypsin, trypsin, elastase, pancreatic kallikrein, and subtilisin
cysteine proteases, including papain, actinidin, rat liver cathepsins B and H.
aspartic proteases, including penicillopepsin, Rhizopus chineses, Endothia acid proteases, and renin;
metalloproteases, including carboxypeptidase and Thermolysin;
unclassified proteases including proteases of unidentified mechanism such as collagenases, aminopeptidases, signal peptidases; and,
exopeptidases and endopeptidases, including aminopeptidases (alpha-aminoacyl peptide hydrolases), dipeptidylpeptidases (dipeptidyl peptide hydrolases), tripeptidylpeptidases (tripeptidyl peptide hydrolases), carboxypeptidases (peptidylamino acid hydrolase), peptidyldipeptidases (peptidyl dipeptide hydrolases such as the angiotensin converting enzyme (ACE) and cathepsin B), dipeptidases, tripeptidases (tripeptide aminopeptidases), and omega peptidases.

Preferred proteases are active under physiological conditions and their activity is not significantly modified by the composition of the polymeric material. In preferred embodiments, the protease added to the polymer specifically degrades the major protein-component of the polymer. The following examples indicate preferred proteases and provide information about their substrate specificity. Bromelain is a plant derived cysteine protease. Chymotrypsin preferentially catalyzes the hydrolysis of peptide bonds involving tyrosine, phenylalanine, and tryptophan. Clostripain (Endoproteinase-Arg-C) is a highly specific sulfhydryl proteinase that hydrolyzes the carboxyl peptide bond of arginine. This protease is particularly useful to regulate the degradation rate of arginine-rich proteins. Collagenase is particularly useful in formulations where collagen is the main component. Elastase is particularly useful in formulations where elastin is the main component. Papain is a sulfhydryl protease of broad specificity. Protease, S. aureus, V8 (Endoproteinase-Glu-C) specifically cleaves peptide bonds on the carboxy-terminal side of either aspartic or glutamic acids. Proteinase K is an endoproteinase with a broad spectrum of action. Subtilisin is a mix of proteases with a wide specificity. Trypsin preferentially catalyzes the hydrolysis of peptide bonds involving lysine or arginine. In one embodiment involving the degradation of an albumin-EDC based sealant, trypsin at 1.2 mg/mL is preferred.

Glucanases

It is also contemplated by the invention that the degradation rate of carbohydrate based sealants or bioadhesives can be regulated. According to the invention including a glucanase in the polymer reaction mixture regulates the degradation of carbohydrate-based polymers. Naturally occurring glucanases are preferred degrading agents for carbohydrate-based polymers. However, modified glucanases are also contemplated by the invention. Modified glucanases include chemically derivatized glucanases and recombinantly modified glucanases. In one embodiment, a modified glucanases with altered proteolytic activity [e.g. increased substrate specificity and catalytic activity] is used to obtain optimal specificity and degradation rate.

Naturally occurring glucanases contemplated by the invention include the following types: agarases, amylases, cellulases, chitinases, dextranases, hyaluranidases, lysozymes, pectinases, alginases (and other preferred enzymes).

Stabilizing Agents

In another embodiment of the invention additives would be used to decrease the rate of degradation. Stabilizing agents contemplated by the invention include the following: enzymatic inhibitors, chelators, allosteric modifiers, substrate-based inhibitors and others known in the art.

Degradation: Application-Specific Considerations

Different rates of degradation are appropriate for different tissue applications, as described herein. Appropriate amounts of enzyme or other degradation agents will be determined by in vivo implantation of the crosslinked gel with different molar ratios of the enzyme or degradation agents. In one embodiment, the sufficient amount of enzyme or other agent is the amount that degrades the sealant, adhesive, or implant at a rate coinciding with the rate of healing.

Methods of the invention are useful to regulate the degradation rate of polymeric compositions at any tissue site in a patient's body. However, in preferred embodiments, the rate of degradation of a polymeric composition is adapted for use at a specific tissue locus. According to the invention, non-limiting examples of a tissue locus are selected from a group comprising connective tissues, endothelial tissues, nervous tissues, and organs. Preferred tissues are selected from the group consisting of bone, skin, cartilage, spleen, renal tissue, hepatic tissues, blood vessels, lung, dural, menengeal, bowel and digestive tissue.

According to the invention, the rate of degradation is optimized to match both the requirements of the use (e.g. bonding tissues or sealing a hole in a tissue) and of the tissue (e.g. CNS, muscle, or liver). For example sealants contemplated by the invention are used to seal fluid leaks in tissues or to bond a first tissue to a second tissue. According to methods of the invention, a fluid or gaseous leak can be sealed by cross-linking the tissues surrounding the leak. Alternatively, a cross-linked gel of the invention can seal a leak by strongly adhering to the surrounding tissue and physically occluding the leak. Preferred methods of the invention are useful for sealing incisions, perforations, and/or fluid or gaseous leaks in biological tissues during a surgical procedure, and comprise contacting the tissue with an effective amount of a sealant preparation along with an appropriate amount of degradation agent under conditions that promote cross-linking of the sealant preparation to the tissue thereby sealing the incision, perforation, or fluid or gaseous leak. Subsequently, the cross-linked polymer is rapidly degraded due to the presence of the degradation agent. In preferred embodiments, the polymer degradation process lasts for a sufficient amount of time to allow the incision or other wound to heal.

Measuring Degradation In Vitro and In Vivo

Gel degradation can be measured in several ways. In general, the rate of gel degradation is measured by monitoring either the disappearance of the starting product, the appearance of degradation product(s), including intermediate products of degradation, or the degradation reaction itself.

Degradation can be measured in vitro by preparing crosslinked gels containing the degradation agent in molds of known volume. The gels are then dialyzed in a low molecular weight cut off dialysis bag against saline and any cofactors necessary for degradation agent activity. It is preferable that conditions be physiologically relevant with regard to pH, temperature and salts. The gel is monitored for a period of several days, with the end point being disappearance of the gel. Compared to control gels with no degradation agents the amount of time for disappearance will be reduced and will vary with the concentration of the degradation agent. In preferred embodiments, the size of the gel is monitored during the degradation assay. Alternatively or additionally, the structural integrity of the gel is monitored during the assay by testing physical properties of the gel at given time intervals.

In a preferred embodiment, gel degradation is monitored spectrophotometrically, For example, for a protein based gel, fluid from the dialysis bag is analyzed by taking spectrophotometric measurements at 214 nm. As degradation proceeds and additional peptides are released, the absorbance at 214 nm will increase.

Degradation can be measured in vivo by preparing crosslinked gels containing the degradation agent in molds of known volume. Pieces of known weight of cured gel can then be implanted into a host animal. Implantation may be intramuscular, but it is preferable to implant the gel at the specific tissue site of interest. Each animal tested is held set numbers of days (usually 15 and 30 days). At the end of the study all implantation, sites are observed macroscopically for the presence of the gel and then explanted and examined microscopically after histological processing (fixed in 10% neutral buffered formalin).

Buffers

In one embodiment of the invention a high buffer concentration in the sealant is used to increase the reactivity of functional groups on the tissue surface that can participate in the bioadhesive/sealant crosslinking reaction. In the case of carbodiimide crosslinkers, the additive optimizes the pH of the tissue surface for reaction with the carbodiimide crosslinker. For example, a protein-based, carbodiimide crosslinked sealant's adhesion to biological tissues (e.g. dura, lung, vascular) can be dramatically improved by adding an acidic buffer to the protein solution. The additive is preferably a buffer with a buffering capacity in the pH range of 4-6.5, in the concentration range of 0.1 to 1 Molar, more preferably pH 5-6 in the concentration range of 0.3 to 0.7M. The most preferred buffer is one that will not interfere in the crosslinking, for example buffers that contain carboxyl groups (e.g. acetate, citrate) or amine groups (Tris-HCl) which will react with carbodiimides or intermediates of the reaction. An example of a non-interfering buffer is morpholino ethanesulfonic acid (MES) and N,N-bis[2-Hydroxyethyl]-2-aminoethane sulfonic acid (BES). The concentration of buffer should be determined experimentally, by analysis of tissue surface pH before and after application, and by examination of adhesion.

In preferred embodiments, the sealant preparation includes a buffer. The pH of the preparation should be compatible with biological tissues, the sealant monomers and the crosslinker. If the crosslinking reaction is pH-dependent, the pH of the preparation should be selected appropriately.

In the embodiments where a buffer is present, the buffer should be effective at the desired pH of the preparation. For example, where EDC is used as the crosslinker, although the pH of the preparation may be between three and ten, the pH is more preferably between about five and about seven, and is most preferably about 6.4. In this highly preferred embodiment, a buffer may be present and capable of maintaining the pH of the solution at or near 6.4. In this particular embodiment, a preferred buffer has a pKa within two pH units of the desired pH (i.e. between 4.4 and 8.4), more preferably within one pH unit of the desired pH, and even more preferably within 0.5 pH units of the desired pH.

If a buffer is present, a preferred buffer will not interfere in the crosslinking reaction. Buffers that contain carboxyl groups (e.g. acetate, citrate) or amine groups (Tris-HCl), for example, may react with carbodiimide crosslinkers, and are less desirable than non-competing buffers that do not contain carboxyl or amine groups. An example of a non-competing buffer is morpholino ethanesulfonic acid (MES). Another preferred buffer is phosphate buffer (e.g. between 10 mM and 250 mM phosphate).

Adhesion Modifiers

A preferred method of the invention comprises providing an additive to modify the adhesiveness of a bioadhesive or sealant composition, a primer solution, or an implant. In one embodiment, the additive is provided to the target surface to increase its chemical affinity for the sealant, primer, or implant. In another embodiment, the additive chemically modifies the molecules of the sealant, the primer solution, or the implant to increase their chemical affinity for the target tissue. This can be accomplished, for example, by modifying the hydrophobicity of the molecules, or their electrochemical properties, or any other physico-chemical property that promotes interaction between the molecules.

Adhesiveness of a surgical sealant, for example, can be assayed by using the sealant to seal an end-to-side anastomosis on blood vessels. By applying pressure to the suture line, the pressure at which a detectable leak occurs can be detected and recorded. This pressure correlates with the adhesiveness of the sealant.

Adhesion of sealants or other compositions to specific surfaces can also be carried out by other methods well known to those skilled in the art. Preferred assays include ASTM tests D903, D1062, D1781, D1876, D3167, D3433, D3762, D3807, and D5041 of the Annual Book of ASTM Standards, published by the American Public Health Association, Inc. of Washington, D.C., the disclosure of which-is hereby incorporated by reference.

Accessory Molecules

According to one embodiment of the invention, an accessory molecule or additive may be used with a sealant or adhesive to modify its physical and/or chemical properties. The type of additive used is determined by the property modification that is most appropriate for a given tissue application. According to the invention, an additive is preferably mixed with a sealant prior to cross-linking or polymerization. As a typical device is prepared by mixing a protein preparation with a cross-linking preparation, then accordingly, the additive may be added to any one of the individual components of the sealant, or to the mixed components immediately prior to polymerization or cross-linking. In another embodiment, the additive is covalently coupled to the protein.

According to the invention, the sealant or adhesive comprises an additive(s) to modify its adhesive, physical and chemical properties. This includes additives that effect: chemical adhesion, viscosity, tensile, elongation, stability to sterilization methods (e.g. gamma irradiation sterilization and electron-beam), solubility, crosslinking, degradation, and wetting. It should be noted that a single additive may effect more than one property, and that multiple additives may have effects on one another. The additive may comprise a molecule selected from the group consisting of viscosity-enhancing agents, cross-linkers, buffers, hydrophilic agents, hydrophobic agents, cationic and anionic agents, hormones, growth factors, anesthetics, antibiotics, surfactants, lipids, fatty acids, anti-inflammatory agents, denaturants, degradation agents, stabilizing agents,

In some preferred embodiments, viscosity-enhancing agents are added to the mixture and, therefore, the concentration of albumin that is employed may be decreased. However, the concentration of albumin is preferably at least 10%, and more preferably at least 20%. In preferred embodiments the viscosity-enhancing agent is itself cross-linked in the reaction. Viscosity-enhancing agents may include substituted or unsubstituted polysaccharides (e.g., glycosaminoglycans or heparin sulfates), fibrous proteins (e.g., collagen, elastin, fibrin, fibrinogen, thrombin, laminin), or other compounds which polymerize under physiological conditions or in the presence of the carbodiimides of the invention (e.g., polyacids and polyamines). Preferred viscosity-enhancing agents include glycosaminoglycans, dextran, hyaluronic acid, collagen, chondroitin sulfate, pectin, carboxymethyl cellulose, alginic acid, elastin, poly(ethyleneglycols) and poly(propyleneglycols).

The viscosity of a protein based surgical sealant or adhesive can be modified by adding a partially cross-linked protein to the formulation. In one embodiment, a protein is polymerized prior to formulation of the sealant or adhesive to increase its molecular weight and thus increase its viscosity.

In another embodiment, the protein is partially cross-linked after formulation of the sealant or adhesive.

Partial cross-linking to increase weight can be accomplished-using any reactant capable of forming a bond between protein molecules, and may include zero-length cross-linkers, bifunctional cross-linkers, and multi-functional cross-linkers. By varying the condition of the partial cross-linking (such as concentration of protein, concentration of cross-linker, reaction rate and time of reaction) the protein solution viscosity can be adjusted for any particular application.

In some preferred embodiments, accessory molecules are added in order to alter the rate and/or degree of cross-linking. In general, a carboxylic acid may reduce the rate or degree of cross-linking by competing with a protein carboxylic group in the first step of the carbodiimide cross-linking reaction. Similarly, an amine may reduce the rate or degree of cross-linking by competing with a protein amine group in the second step of the carbodiimide cross-linking reaction. However, polycarboxylic acids, polyamines, poly(carboxy/amino) compounds (i.e., compounds having a multiplicity of carboxyl and amino groups), and mixtures thereof, may increase the rate of gel formation by reacting with carbodiimides to form cross-links with two or more protein molecules, thereby participating in the gel formation. Such polycarboxylic acids, polyamines, and/or poly(carboxyl/amino)compounds should have a relatively high density of carboxy and/or amino groups.

In further embodiments, the hydrophobicity of the albumin solution is increased by solubilizing the albumin in a solution that is more hydrophobic than water. In a preferred embodiment, the albumin is solubilized in a solution comprising a secondary or tertiary alcohol. Preferably, albumin is provided in a solution of isopropyl alcohol (IPA) or isobutyl alcohol (IBA). Most preferably, a 30% solution of BSA is prepared with 20% IPA or 8% IBA.

In another aspect, the invention provides methods and compositions that bind or adhere to synthetic material such as artificial blood vessels (for example PTFE material) or biological implants (for example polyethylene material).

In another aspect, the invention provides a kit for producing a bioadhesive, surgical sealant or implantable device comprising, in separate containers, an albumin preparation, and a carbodiimide preparation. In a preferred embodiment, the kit further comprises an accessory molecule, preferably a molecule selected from the group consisting of viscosity-enhancing agents, cross-linkers, buffers, hormones, growth factors, antibiotics, anesthetics, anti-inflammatory agents, hydrophobicity increasing agents, and surfactants.

In some embodiments, the albumin solution being cross-linked comprises additional reagents to promote interaction with the tissues at the site of application.

Mixing and Delivery of Biopolymer-Crosslinker Solutions

According to the invention the mixing of a protein component with the crosslinker component can occur just prior to the application by simple end to end syringe mixing through a connector. In another embodiment a binary delivery system, having separate compartments holding the protein component and the cross-linker component prior to dispensing and mixing, may be particularly useful. Thus, in one method, a double-barreled syringe that simultaneously dispenses and mixes the components is used. Such a double-barreled syringe may be quite convenient for in vivo applications where the bioadhesive or surgical sealant is applied to the site of tissue injury or incision.

In another embodiment of the invention, a double barrel system comprises a first barrel containing a protein solution at a pH near or below where the cross-linking reaction may occur (e.g., pH 5.0-6.0 for carbodiimide crosslinking), and a second barrel containing the protein solution at alkaline pH (i.e. pH≧8) sufficient to slow crosslinking to an extended period of time. The crosslinker can then be mixed with this second component and remain useable in a fluid state. Upon mixing the low and high pH solutions crosslinking proceeds. In this embodiment, the pH and/or buffer systems in the two barrels must be selected such that, upon mixing, the pH of the resultant solution is optimized to permit the cross-linking reaction to proceed efficiently.

In an alternative embodiment, a single barreled syringe contains the protein solution separated from a crosslinker by a breakable membrane. The crosslinking reaction is started by breaking the membrane, and the resulting mixture is applied as described above. In another embodiment, the crosslinker is encapsulated within a microsphere which, when shear forces are applied, rupture allowing the crosslinker to mix with the sealant solution. Alternatively, the two components may be applied as a spray from a device with separate reservoirs for the two components. Finally, although it is not preferred, the two components may be applied sequentially. This method suffers from the disadvantage that the components will not be as thoroughly mixed, and only a thin coat of cross-linked protein may form at their interface.

Primers

Methods of Priming Tissues to Promote Adhesion of Sealants and Adhesives

The present invention also provides methods for preparing a tissue to react with a protein based tissue sealant or adhesive. According to the invention primers are used to promote adhesion between a tissue substrate and a sealant or adhesive. In one embodiment, the primer optimizes the tissue-sealant interface by matching one or more chemical and/or physical properties of the tissue surface to that of the sealant/bioadhesive. In one embodiment a primer washes a tissue to remove any weak boundary layers at the surface. In another embodiment, the primer will contain molecules that strongly bind to the tissue and will subsequently react and bind with the sealant. For example, a primer containing perfluorooctanoic acid (PFOA) improves adhesion of the sealant to expanded PTFE. The fluorinated tail binds strongly to expanded PTFE while the free carboxyl group can react with the sealant. In another embodiment the primer will optimize the tissue surface to participate in the crosslinking reaction of the bioadhesive or sealant. In another embodiment the primer may do any combination of things listed above. A primer is generally applied using a brush, sprayer, or by simple irrigation.

Primers are used to promote adhesion between a tissue substrate and a device. In one embodiment, the primer optimizes the tissue-sealant interface by matching one or more chemical and/or physical properties of the tissue surface to that of the sealant/bioadhesive. In one embodiment a primer washes a tissue to remove any weak boundary layers at the surface. In another embodiment, the primer will contain molecules that strongly bind to the tissue and will subsequently react and bind with the sealant. In another embodiment the primer will optimize the tissue surface to participate in the crosslinking reaction of the bioadhesive or sealant. In another embodiment the primer may do any combination of things listed above. A primer is generally applied using a brush, sprayer, or by simple irrigation.

Cleansing a Tissue Surface

Each tissue surface has differing properties (e.g. type of bodily fluids present, hydrophobicity, hydrophilicity, pH, and charge) thus, how a particular sealant interacts with the surface will differ among the various tissues to which they are applied. These fluids and/or chemical characteristics form a weak boundary layer that will interfere with the adhesion of the sealant to that surface and are preferably removed. A primer may be used to wash a tissue site prior to application of a surgical sealant to remove body fluids that could interfere with the sealant. A primer is generally a solution that is brushed, sprayed, or irrigated onto a tissue. An additive can be mixed with the primer solution to improve its compatibility with the tissue surface and ultimately improve washing efficiency. For example a lung sealant application may require a hydrophobic and/or low pH primer to effectively remove the pleural fluid that bathes the lung surface. The primer could be an acidic solution of IPA (20%), or it may contain an artificial lung surfactant such as tyloxapol and dipalmitoylphosphatidylcholine.

In the case of synthetic tissue (e.g. expanded PTFE) the primer should lower the surface free energy of the substrate and subsequently the contact angle of the sealant. One example of this is priming the substrate with a solution of a perfluorinated compound such as perfluorooctanoate, or Zonyl FSN (Dupont). It will be recognized by any one of ordinary skill in the art that the choice of primer additive will depend on the composition of the particular tissue.

Primer Interaction with Tissue or Sealant

According to the invention the primer may contain a molecule that will strongly bind to a tissue substrate. This strongly bound molecule should also have a high affinity for the sealant, or contain functional groups that can participate in the sealant's crosslinking reaction. For example, the primer could be perfluorooctanote for a carbodiimide crosslinked sealant on synthetic tissue (e.g. expanded PTFE). The fluorinated tail strongly interacts to the expanded PTFE, while the carboxyl group can react with the sealant via carbodiimide crosslinking.

According to the invention the primer may contain a molecule that will strongly bind to a tissue substrate. This strongly bound molecule should also have a high affinity for the sealant, or contain functional groups that can participate in the sealant's crosslinking reaction. For example, the primer could be perfluorooctanote for a carbodiimide crosslinked sealant on synthetic tissue (e.g. expanded PTFE). The fluorinated tail strongly binds to the expanded PTFE, while the carboxyl group can react with the sealant via carbodiimide crosslinking.

Modification of the Tissue Surface

According to the invention an additive is mixed with the primer solution to modify the tissue-sealant interface. In one embodiment the primer solution is a dilute solution of a crosslinker. For example, a protein-based sealant's adhesion can be improved if the tissue substrate is first primed either with a carbodiimide, carbodiimide/Sulfo-N-hydroxysuccinimide, glutaraldehyde or any combination thereof. One of ordinary skill would recognize that any crosslinking solution would produce similar results providing the tissue surface conditions were optimized for the reaction it catalyzes. The concentration of the crosslinker in the priming solution depends on the reactivity and lifetime of the crosslinker. For example carbodiimide intermediates are short lived, while reactive esters and glutaraldehyde are longer lived. The concentration of crosslinker may be determined by a series of priming experiments using either in vitro burst models or in vivo testing on the tissue. The preferred concentration of the carbodiimide solution is between 1% and 50% wlw, more preferably between 10% and 30% w/w, and most preferably between 15% and 25% w/w. The preferred concentration of carbodiimide and sulfoNHS are between 1% and 50% for each, more preferably 10 and 30%. For a glutaraldehyde primer the concentration can be between 0.1 and 10%, preferably between 0.5 and 5% and most preferably between 0.7 and 1.3%.

In another preferred embodiment, the primer solution modifies the tissue surface to participate in the crosslinking reaction of the sealant. The primer optimizes the tissue surface to the crosslinker being used (e.g. pH). For example, a carbodiimide crosslinked device's adhesion can be dramatically improved by priming the tissue with a buffer solution to “activate” it. Activate is defined as optimizing the surface chemistry of the tissue so functional groups present at the surface can participate in the crosslinking reaction. The primer can be as simple as a dilute HCl solution, but is preferably a biocompatible buffer with a pK in the range of 4-7. The most preferred buffer is one that will not interfere in the crosslinking reaction; such as morpholino ethanesulfonic acid (MES). If it becomes necessary to use a buffer that contains groups that interfere with the crosslinking, for example buffers that contain carboxyl groups (e.g. acetate, citrate) or amine groups (Tris-HCl), which will react with carbodiimides or intermediates of the reaction, then priming may be done in two steps; the surface is primed with a first buffer at a high concentration, followed by water or the same buffer at a lower concentration dilute enough not to interfere with the crosslinking reaction. The concentration of the buffer is dependent on the volume of buffer to be used. For example, a dilute solution of buffer will require more volume to change the pH than a higher concentration of the same buffer. In one embodiment of the invention the concentration of the buffer is between 0.01 and 2 M. The preferred concentration is between 0.1 and 1 M, and most preferably between 0.3 and 0.7 M.

In the case of crosslinkers that function at a basic pH the buffers should increase the pH of the surface, while not interfering with the crosslinking reaction. For example, the adhesion of a polycarboxylated-based, reactive ester crosslinked sealant can be improved by priming the tissue with a buffer that has a basic pK (>7) such as BES.

According to the invention, an additive is mixed with the primer solution to modify the tissue-sealant interface. In one embodiment the primer solution is a dilute solution of a crosslinker. For example, a protein-based sealant's adhesion can be improved if the tissue substrate is first primed either with a carbodiimide, carbodiimide/Sulfo-N-hydroxysuccinimide, glutaraldehyde or any combination thereof. Of course it is recognized in the art that any crosslinking solution would produce similar results providing the tissue surface conditions were optimized for the reaction it catalyzes. The concentration of the crosslinker in the priming solution depends on the reactivity and lifetime of the crosslinker. For example carbodiimide intermediates are short lived, while reactive esters and glutaraldehyde are longer lived. The concentration of crosslinker may be determined by a series of priming experiments using either in vitro burst models or in vivo testing at the tissue in question. The preferred concentration of the carbodiimide solution is between 1% and 50% w/w, more preferably between 10% and 30% w/w, and most preferably between 15% and 25% w/w. The preferred concentration of carbodiimide and sulfoNHS are between 1% and 50% for each, more preferably 10 and 30%. For a glutaraldehyde primer the concentration can be between 0.1 and 10%, preferably between 0.5 and 5% and most preferably between 0.7 and 1.3%.

Kits

According to the invention a useful kit for producing a protein-based tissue adhesive or sealant comprises: (1) a tissue primer (preferably a morpholinoethanesulfonic acid buffer), (2) a protein preparation (preferably albumin) (3) at least one preparation selected from a surfactant preparation and a lipid preparation (preferably tyloxapol and dipalmitoylphosphatidylcholine) (4) a cross-linker preparation (preferably carbodiimide), and (5) a preparation of protein degrading agent (preferably trypsin).

In another embodiment a kit for producing a protein-based tissue adhesive or sealant comprises: (1) a protein preparation, (2) at least one preparation selected from a surfactant preparation or a lipid preparation, and (3) a cross-linker preparation, and that may further comprise at least one preparation selected from: (a) a tissue primer, and (b) a preparation of protein degrading agent.

In another embodiment, a kit for producing a protein-based tissue adhesive or sealant comprise: (1) a protein preparation, (2) a cross-linker preparation.

In a further embodiment, a kit for producing a protein-based tissue adhesive or sealant comprise: (1) a protein preparation, (2) a preparation of protein degrading activity, and (3) a cross-linker preparation.

According to a preferred embodiment of the invention, a useful kit comprises at least two of the following: a primer, a primer applicator (e.g. a brush), a protein preparation, a crosslinker preparation, a crosslinker diluent, a degrading agent, a degrading agent diluent, a syringe connector, a sealant applicator, or printed instructions describing proper uses of the kit. Preferred kits comprise at least four of the above components. Highly preferred kits comprise at least six of the above components. The most preferred kits comprise at least eight of the above components.

A preferred kit provides a first protein preparation at an acidic pH, preferably between about 3.0 and 6.0, and a second protein preparation at a basic pH, preferably between about 6.5 and 10.0. In a preferred embodiment, an EDC crosslinker is mixed with the second protein preparation. When the first and second protein preparations are mixed, crosslinking is initiated, because the resulting pH is optimal for crosslinker activity.

Methods for Priming and Sealing Tissues

Using an Additive for Improved Leak Sealing in Lung

A preferred method of the invention comprises providing an additive to a sealant for use in a lung. The additive modifies the surface tension and hydrophobicity of the sealant mixture to promote interaction between the sealant and the surface of the lung tissue. Preferably, in the presence of the additive, the sealant mixture spreads evenly over the surface of the lung tissue.

In a preferred embodiment, an albumin solution comprises a surfactant and/or a lipid when it is used as a pulmonary sealant. Preferably, the surfactant and lipid component is similar to the natural surfactant and lipid composition of the lung. Alternatively, synthetic surfactants and lipids may be used.

Methods of Bonding or Sealing Fluid or Gas Leaks in Tissue

The invention also describes methods for using the preferred compositions of this invention as surgical sealants and adhesives. According to the invention, one method for bonding or sealing fluid or gas leaks in tissue comprise the steps of mixing a preferred composition (such as albumin, tyloxapol, and dipalmitoylphospatidylcholine) with a crosslinker capable of crosslinking the protein and then applying the sealant to a tissue wound, thereby to bond the tissue or seal a fluid or gas leak in the tissue.

Another method for bonding or sealing fluid or gas leaks in tissue comprise the steps of applying to the tissue locus a preferred composition and a crosslinker and permitting the preparation to form crosslinks, thereby to bond said tissue or seal a fluid or gas leak in said tissue.

Using an Additive for Improved Leak-Sealing in Synthetic Tissue

A preferred method of the invention comprises providing an additive to a sealant or bioadhesive for use with synthetic material. In one embodiment, the additive modifies the surface tension of the sealant/bioadhesive to match that of the synthetic material, thereby promoting adhesion. In alternative embodiments, the additive modifies the hydrophobicity or provides a moiety that specifically binds to the synthetic material. The additive is preferably covalently bound to the sealant monomers prior to or during the cross-linking reaction of the sealant or adhesive.

Analytical Methods

A number of analytical methods are employed in the present invention to monitor and optimize the effect of additives and measure the usefulness of the device for particular applications. The methods referred to in the invention are outlined below.

In Vitro Assays

Adhesion

Adhesion of a surgical sealant or bioadhesive is defined as how well it bonds to a tissue substrate. One method of measuring a sealant's adhesion is to conduct a burst test. Two types of tests are outlined below.

Dural Burst Test

Defects including incisions, flaps, and expanded-polytetrafluoroethylene (expanded PTFE)patches are made in porcine dura. The dura is then placed in a apparatus, the sealant is applied, the chamber of the apparatus is filled with saline and pressurized. The pressure at which a detectable leak occurs (air bubbling in saline) can be measured and recorded. This pressure correlates with the adhesiveness and effectiveness of the sealant or bioadhesive.

Vascular Burst Test

End-to-side anastomosis on blood vessels or expanded PTFE grafts are treated with sealant and then pressurized using an aqueous dye solution. The pressure at which a detectable leak occurs (release of aqueous dye) can be measured and recorded. This pressure correlates with the adhesiveness and effectiveness of the sealant or bioadhesive.

Skin Adhesion Test for Seroma

In addition to the burst test, adhesion of sealants or other compositions to specific surfaces can also be carried out by other methods well known to those skilled in the art. Preferred assays include ASTM tests D903, D1062, D1781, D1876, D3167, D3433, D3762, D3807, and D5041 of the Annual Book of ASTM Standards, published by the American Public Health Association, Inc. of Washington, D.C., the disclosure of which is hereby incorporated by reference.

Rheometric Properties

Viscosity and other rheometric properties (flow, effect of shear) were measured using a Brookfield rheometer.

Tensile and Elongation

Tensile and elongation were measured using a Chatillon force gauge.

Cure Time is a measurement of the time required for a sealant or bioadhesive to go from a liquid phase to a solid (or semisolid phase). In these experiments it is defined as the point at which the force required to extrude the material through an orifice rises exponentially.

Wetting was measured using contact angle. Contact angle was determined using a goniometer.

In Vivo Assays

Lung Sealant Assays

Partial Lobectomy
A partial lobectomy model was created by removing an approximately 1-3″ section of lung tissue from an edge of a lobe. This created a 1-3″ long by ¼-½″ wide exposed-parenchyma wound site. Wound site hemostasis was achieved using electrocautery. Air leaks from the wound site were verified before treatment by submerging the wound site in saline and inflating the lung. Air leaks were verified by the presence of air bubbles in the saline. The lung was then deflated for sealant application.

Planar Dissection
A planar dissection model was created by removing a section of pleural tissue from the planar surface of the lung. The approximately ½″ diameter by ⅛″ deep exposed-parenchyma wound site was made using forceps and a standard electrocautery instrument. Wound site hemostasis was achieved using electrocautery. Air leaks from the wound site were verified before treatment by submerging the wound site in saline and inflating the lung. Air leaks were verified by the presence of air bubbles in the saline. The lung was then deflated for sealant application.

Wedge Resection
A wedge resection model was created by removing a V-shaped section of lung tissue from the edge of a lobe. An approximately ½″ incision was made into the edge of a lobe at a 45 degree angle using surgical scissors. A second ½″, 45 degree angle incision was made, meeting the first to form a “V”, and the V-shaped section of tissue removed. This left an exposed-parenchyma wound site. Wound site hemostasis was achieved using electrocautery. Air leaks from the wound site were verified before treatment by submerging the wound site in saline and inflating the lung. Air leaks were verified by the presence of air bubbles in the saline. The lung was then deflated for sealant application.

Staple Line
A staple line model was created using a standard staple line instrument. An approximately 3″ incision into the side of a lobe. Every second staple in the staple line was removed. Wound site hemostasis was achieved using electrocautery. Air leaks from the wound site were verified before treatment by submerging the wound site in saline and inflating the lung. Air leaks were verified by the presence of air bubbles in the saline. The lung was then deflated for sealant application.
   Vascular Sealant Assays

Suture Line
A suture line model was created by making an incision in a blood vessel approximately 1 cm in length and suturing. The suture spacing could be varied to increase the level of bleeding. Hemostasis was achieved using clamps or loops. A sealant was considered effective if no visible bleeding occurred after removal of the clamps.

Synthetic Patch.
The synthetic patch model was created by sewing a 2×20 mm expanded patch into the blood vessel. The sealant is used to cover the patch to obtain a fluid tight seal.

End to Side Anastomoses
In order to create a vascular anastomosis that would leak consistently, a hemorrhage model was developed by administering anticoagulants, employing hemodilution and increasing the space between sutures from 1 mm (normally used) to ˜4 mm. Using this model, the following configurations of anastomoses were created: 1) arterial suture line, 2) end-to-side with autogenous vein, and 3) end-to-side with an expanded PTFE graft. As expected, significant bleeding was observed in 100% of the anastomoses after using sutures only (N=35). These leaking anastomoses were assigned into either the treated (vascular sealant) group or the control (thrombin-soaked gelatin sponge) group. The number of leak-free anastomoses and the time to hemostasis were recorded for both groups.
The efficacy of the sealant was proven in terms of time to hemostasis and the number of anastomoses sealed. The configuration of the anastomosis did not affect efficacy.
   Dural Sealant Assays

Incision
Following a craniotomy an incision was made in the dura of about 1 cm in length and leakage of cerebrospinal fluid was detected. Fluid stasis was achieved by tilting the animal and the sealant would be applied. A sealant was considered effective if no visible leaking could be detected
   Seroma Prevention Assays
Seroma prevention can be analyzed using rats that undergo a lymphadenectomy. The wound is dried with sterile gauze, treated with sealant or bioadhesive, and closed using sutures. Animals are evaluated 7 days post operatively for serous drainage, adhesion formation and histology.

### EXAMPLES

The following non-limiting examples demonstrate the use of preferred compositions and methods outlined above to form bioadhesives, sealants and implants.

### Example 1

### Examples of Preferred Compositions

According to the invention one useful sealant formulation consists of aqueous bovine serum albumin (BSA), tyloxapol, and dipalmitoylphosphotidylcholine (DPPC). The albumin concentration can be between 15 and 55 w/w %, but is preferably between 25 and 45 w/w % and most preferably between 30 and 40 w/w %. The tyloxapol is added to increase viscosity, and disperse the insoluble DPPC. The concentration of tyloxapol can be between 0.05 and 15 w/w %, but is preferably between 3 and 10%. The DPPC is added to increase hydrophobicity and interaction with hydrophobic tissue, as well as increase elongation properties of the final sealant. The concentration of DPPC can be between 0.5 and 10 w/w %, but is preferably between 3 and 8%. The pH of the final solution can be between 4.5-8.0, but is preferably between 5 and 7, and most preferably between 6 and 7. The overall strength of the lung sealant will depend on the final albumin concentration, crosslinking density, and effects of the additives.

Another useful sealant formulation consists of aqueous bovine serum albumin (BSA), and sodium dodecylsulfate (SDS). The albumin concentration is similar to the previously described formulation. The SDS is added to increase viscosity and hydrophobicity. The concentration of SDS can be between 0.5 and 10 w/w %, but is preferably between 1 and 7%, and most preferably between 2 and 5%.

Another useful sealant formulation consists of aqueous bovine serum albumin (BSA), and sodium octanoate. The albumin concentration is similar to the previously described formulation. The sodium octanoate is added to increase hydrophobicity. The concentration of sodium octanoate can be between 0.5 and 15 w/w %, but is preferably between 3 and 10%, and most preferably between 5 and 8%.

Another useful sealant formulation consists of collagen derivatized with glutaric anhydride and perfluorooctanoic acid (PFOA). The collagen has been derivatized with glutaric anhydride. The derivatization is to increase the solubility of collagen at physiological pH and can be between 5 and 95% but is preferably between 10 and 60% and most preferably between 25 and 45%. The derivatized collagen concentration can be between 2 and 15 w/w %, but is preferably between 5 and 10%. The PFOA is added to increase wetting and adhesion to expanded PTFE. The concentration is between 0.05 and 5 w/w %, but is preferably between 0.2 and 2%, and most preferably between 0.5 and 1%.

Another useful sealant composition consists of aqueous bovine serum albumin (BSA), poly(ethylene glycol 600) (PEG 600), perfluorooctanoic acid (PFOA). The albumin concentration can be between 15 and 55 w/w %, but is preferably between 25 and 45 w/w % and most preferably between 30 and 40 w/w %. The PEG 600 concentration can be between 0.5 and 20 w/w %, but is preferably between 10 and 20 w/w %. The PFOA concentration can be between 0.5 and 10 w/w %, but is preferably between 1 and 7 w/w % and most preferably between 2 and 4 w/w %. The pH of the final solution can be about 4.5-8.0, but is preferably about 5-7 and most preferably about 6-7.

Another useful sealant composition consists of aqueous bovine serum albumin (BSA), pectin, perfluorooctanoic acid (PFOA). The albumin concentration can be between 15 and 55 w/w %, but is preferably between 25 and 45 w/w % and most preferably between 30 and 40 w/w %. The pectin concentration can be between 0.5 and 20 w/w %, but is preferably between 1 and 10 w/w % and most preferably between 2 and 4 w/w %. The PFOA concentration can be between 0.5 and 10 w/w %, but is preferably between 1 and 7 w/w % and most preferably between 2 and 4 w/w %. The pH of the final solution can be about 4.5-8.0, but is preferably about 5-7 and most preferably about 6-7.

Another sealant formulation consists of perfluorooctanoic acid (PFOA) derivatized albumin. The albumin is derivatized with PFOA, which will increase viscosity and wetting of sealant into expanded PTFE. The substitution can be between 0.5 and 95%, but is preferably between 5 and 50% and most preferably between 10 and 30%. The derivatized albumin concentration can be between 5 and 40 w/w %, but is preferably between 20 and 35%.

### Example 2

### Demonstrating the Regulation of Degradation

I. Assaying Carbodiimide Cross-Linked Albumin Degradation In Vitro

A protein solution (300 μl) was mixed with a solution of trypsin (20 μl). The concentrations of the enzyme are in a certain relation to the concentration of the protein. The resulting solution is then mixed with 31 μl of a 250 mg/ml solution of 1-ethyl-3-(3-dimethylaminopropyl)carbodiimide-HCl (EDC). The protein-crosslinker-trypsin solution was poured into molds and allowed to set for 5 minutes. The resulting 10×3 mm cylinders are removed from the molds and placed in a 12 kDa molecular weight cut off dialysis bag. The bags are placed in 4L of 0.9% NaCl supplemented with 1 mM CaCl₂ with constant stirring and maintained at 37° C. The samples are periodically observed and degradation was equated to the disappearance of the cylinder.

Experiment 1:

SAMPLES: A 30% BSA solution pH 5.5 was mixed with a 225 mg/ml solution of trypsin (the enzyme was obtained from Sigma Chemical Corp. and had a S.A=3100 USP Units/mg of protein). The final mixture corresponds to a 20:1 (protein:enzyme) weight ratio. Control samples were made by replacing the trypsin solution with water.
RESULTS: The control degraded in 56 days, while the sample containing trypsin disappeared in only two (2) days.
   Experiment 2:
SAMPLES: The same BSA and trypsin samples were used. The amount of trypsin added was lowered resulting in weight ratios of 50:1, 100:1, 200:1,and 500:1 BSA:trypsin (same enzyme source as in Experiment 1). Control contained water instead of enzyme.

RESULTS: The experimental results, summarized in the following table, show that, again the cured gel that contained the enzyme disappeared before the control. The results also show that by changing the protein:enzyme ratio we can regulate the rate of degradation within the gel.

| BSA:Trypsin Ratio | Degradation Time |
|---|---|
| (Units) | (Days) |
| 50:1 (5580) | 5 |
| 100:1 (2790) | 7 |
| 200:1 (1395) | 9 |
| 500:1 (558) | 16 |
| Control | 41 |

(The values in parenthesis indicate the total amount of units of enzyme added to the sample. units are standard USP units for trypsin)
Experiment 3:
SAMPLE: A solution of 35% BSA containing 5% Tyloxapol and 5% DPPC in 100 mM MES buffer at pH 6.3 was mixed with varying amounts of trypsin (obtained from Worthington Biochemical Corp. S.A.=3458 U/mg). The EDC solution was less concentrated (200 mg/ml) but was mixed in the same ratios. Controls contained water instead of enzyme solution.

RESULTS: The degradation times for these molds are shown in the following table:

| Protein:Enzyme | |
|---|---|
| Ratio | Degradation Time |
| (Units) | (Days) |
| 12.5:1 (48412) | 0.8 |
| 31:1 (19365) | 2 |
| 63:1 (9682) | 3.7 |
| 125:1 (4841) | 6 |
| 310:1 (1937) | 8 |
| Control | 35 |

Again we observe a shorter half-life of the mold that contains more enzyme, as compared to those with less enzyme, and controls without trypsin.
Experiment 4:
SAMPLE: The BSA, tyloxapol and DPPC concentrations are the same as in the previous experiment. The buffer utilized was also the same, except that it also contained 1 mM CaCl₂, which functions as a stabilizer of the trypsin molecule and facilitates the activation of any trypsinogen present in the enzyme solution. Trypsin was obtained from Intergen Corp., and the sample had a S.A.=3194 USP U/mg.

RESULTS: The degradation of these samples is presented in the following table:

| Protein:Enzyme | |
|---|---|
| Ratio | Degradation Time |
| (Units) | (Days) |
| 1966:1 (284) | 2 |
| 3889:1 (144) | 3 |
| 7609:1 (73) | 5 |
| 15909:1 (35) | 12 |
| 38889:1 (14) | 17 |
| Control | 35 |

The differences observed among the samples is due mostly to the changing conditions. In experiments 1 and 2 the crosslinking density is higher resulting in a more resistant control, and the need for more enzyme as compared to experiments 3 and 4. The latter differ from each other in the amounts of enzyme included in the gel and the presence of calcium which stabilizes the enzymes and activates any proenzyme present.
II. Assaying Carbodiimide Cross-Linked Albumin Degradation In Vivo

Cross-linked protein molds again were made containing different amounts of trypsin. These were then implanted into rabbits and the tissue reaction was observed both macroscopically as well microscopically.

Preparation of the Implant:

All materials were prepared sterile. A 30% rabbit serum albumin (RSA)(Sigma Chemical Corp., St. Louis, Mo.) solution was prepared at pH 5.7. A 0.5 ml aliquot of this solution was mixed with a 20 μL aliquot of a trypsin solution (1.5-150 mg/ml) (Sigma Chemical Corp., St. Louis, Mo.). This solution was then mixed with a tenth volume of the crosslinking solution, EDC 250 mg/ml (Sigma Chemical Corp., St. Louis, Mo.). The gel was deposited in cubic molds (10×10×1 mm) and allowed to cure for 5 minutes.

The trypsin concentrations used and the protein:enzyme weight ratios obtained were:

1. 150 mg/ml for a 50:1 ratio

2. 15 mg/ml for a 500:1 ratio

3. 1.5 mg/ml for a 5000:1 ratio

Implantation Method:

Pieces of the cured gel were implanted into the paravertebral muscle of New Zealand white rabbits. From the cubes strips measuring 1×1×10 mm were aseptically cut. Each animal received three intramuscular implants, corresponding to each gel. For each gel one animal was held for 15 days and another for 30 days. At the end of each time period the implantation sites were observed macroscopically and scored for hemorrhaging, erythema, necrosis, purulence and encapsulation. The sites were then retrieved and examined microscopically after histological processing. The table summarizes the results.

| Albumin:Trypsin Ratio | Days | Macroscopic Observation |
|---|---|---|
| 50:1 | 15 | No material observed at site |
| | 30 | No material observed at site |
| 500:1 | 15 | Trace material observed |
| | 30 | No material observed at site |
| 5000:1 | 15 | Material observed at site |
| | 30 | No material observed at site |

Histopathology of the implant sites showed that the microscopic observations correlate with these results. The 50:1 dilution had a very limited inflammatory response at 15 and 30 days, while the 5000:1 implantation sites presented increased inflammation and microscopic particles of the cured gel. The 500:1 sites showed an intermediate response, with a decrease in inflammation and implant from 15 to 30 days.

III. Assaying Carbodiimide Cross-Linked Carbohydrate Degradation In Vitro

Plant derived carbohydrates are not very susceptible to degradation in the human body. A classic example is cellulose, which cannot be degraded or metabolized in the body, and if implanted would have to be small or would have to be degraded by enzymes supplied with the implant. To utilize a derivatized form of cellulose as an implant, we incorporated cellulase, which are enzymes capable of degrading cellulose, into the formulation to affect the degradation rate if the sealant.
SAMPLE: A solution was made that contained 16.5% Carboxymethylcellulose (CMC) and 2.5 mol % Polyoxyethylene bis(Amine) at a final pH of 6.3. To affect the degradation of the crosslinked solution, it was mixed with varying amounts of cellulase (Worthington Biochemical Corp.) that had a S.A.=62.9 Units/mg. To crosslink the solution a 400 μL aliquot of this solution is mixed with 100 μL of a solution containing 44 mg of EDC and 4.4 mg of N-hydroxysulfosuccinimide (the crosslinker solution). Both were mixed and applied into the same cylindrical mold described previously and allowed to cure for 3 minutes.
   The cylinders were put into dialysis tubing (12 kDa MWCO) and left in a 0.9% NaCl solution bath at 37° C., with constant stirring. The cylinders were observed on a daily basis and degradation was equated to the disappearance of the crosslinked cylinder.

RESULTS: The following table illustrates the degradation of the crosslinked CMC gels containing cellulase:

| CMC:Cellulose | |
|---|---|
| Ratio | Degradation Times |
| (Units) | (Days) |
| 50:1 (84) | 0.05 |
| 100:1 (42) | 0.05 |
| 200:1 (21) | 0.75 |
| 500:1 (8.4) | 1.00 |
| 1000:1 (4.2) | 1.70 |
| 2000:1 (2.1) | 5 |
| 5000:1 (0.84) | 16 |
| 10000:1 (0.42) | 40 |
| Control | >60 |

The control has continued past 60 days, but what is significant is the dramatic effect that the inclusion of cellulase has on the degradation of the crosslinked gel. Only small amounts of enzyme need to be incorporated to rapidly degrade the gel, which may be due to the large amount of sites that are available for the enzyme(s).

### Example 3

### Preparation of a Protein-Surfactant-Lipid Sealant Composition

A sealant composition based on 35% albumin, 5% tyloxapol, and 5% dipalmitoylphosphotidylcholine (DPPC) is prepared as follows: For a 100 g scale, 5 g of Tyloxapol (Sigma) is dissolved in 55 g water, followed by 5 g of DPPC (Genzyme). The solution is stirred until the DPPC is well dispersed. To this solution 35 g of bovine serum albumin (Intergen) is slowly added and mixed until fully dissolved. The pH of the final solution is adjusted to 6.3-6.6 using 6N HCl.

### Example 4

### Preparation of a Protein-Surfactant Sealant Composition

A sealant composition based on 35% albumin, 5% tyloxapol, and 4.5% perfluorooctanoic acid (PFOA), and 0.5% morpholinoethanesulfonic acid (MES) is prepared as follows: For a 100 g scale, 5 g of Tyloxapol (Sigma) is dissolved in 55 g water, followed by 0.5 g of MES and 4.5 g of PFOA (Aldrich). The solution is stirred and titrated with 6N HCl to a pH of 5.5-6.0. To this solution 35 g of bovine serum albumin (Intergen) is slowly added and mixed until fully dissolved. The pH of the final solution is adjusted to 6.3-6.6 using 6N HCl.

### Example 5

### Preparation of a Protein-Surfactant Sealant Composition

A sealant composition based on 7% collagen and 0.5% perfluorooctanoic acid (PFOA) is prepared as follows for a 100 g scale: 0.5 g of PFOA is suspended in 92.5 g of 25 mM phosphate buffer and pH is adjusted to 5-6 using 10 M NaOH. This is followed by the addition of 7 g of 20-45% derivatized collagen (derivatized with glutaric using methods known in the art). The resulting solution is then titrated to a pH of 6.5-7.5 using 10M NaOH.

### Example 6

### Preparation of a Protein-Surfactant-Viscosity Modifier Composition

A sealant composition based on 36.6% albumin, 2.5% perfluorooctanoic acid (PFOA), 15.2% poly(ehtyleneglycol (600)), and 0.9% MES is prepared as follows: For a 100 gram scale, 2.5 g PFOA, 0.9 g MES and 15.2 g PEG 600 are dissolved in 44.8 g water and titrated with 10 M NaOH to a pH of about 6.5. To this solution 36.6 g of bovine serum albumin (Intergen) is slowly added and mixed until fully dissolved.

A sealant composition based on 40% albumin, 2.8% perfluorooctanoic acid (PFOA), 2.3% Pectin, and 0.9% MES is prepared as follows: For a 100 gram scale, 2.8 g PFOA, 0.9 g MES and 2.3 g pectin are dissolved in 54 g water and titrated with 10 M NaOH to a pH of about 6.2. To this solution 40 g bovine serum albumin (Intergen) is slowly added and mixed until fully dissolved.

### Example 7

### Use of a Two Component Sealant System Using Carbodiimide as Crosslinker

Two sealant compositions similar to example 1 were prepared, but the first solution was made up in 50 mM phosphate and the pH was adjusted to pH 8.1 (part I). The second solution was made up in 0.5M MES and the pH was adjusted to 5.4 (part II). 1.8 mL of part I was mixed with 0.2 mL of 40% w/v EDC resulting in an activated solution of part I. This material will remain liquid for 15 minutes. The 2.0 mL of active part I solution was then mixed equally with part II through a static mixer nozzle. The resulting mixture rapidly gelled (in about 18 seconds).

### Example 8

### Effect of Different Primers on Adhesion to Lung Tissue

A series of studies were conducted to determine an effective primer system for lung tissue. Experiments were carried out on an anesthetized dog. The first experiments were conducted on a model for thoracic surgical complications on the lung including pulmonary air leaks.

Wound sites were made at various points on the lobe of the lung using scissors. The wound resulted in bleeding and loss of air. Hemostasis was achieved by cauterization and an air leak was confirmed as evidenced by air bubbles from the submerged lung.

One single formulation was used for all experiments. The formula consisted of BSA (35%), tyloxapol (5%), DPPC (5%), and EDC-HCl (2%).

Priming was done using a brush or by spraying (the method used will be indicated). The following Table outlines the results.

| | Application | |
|---|---|---|
| Primer Type1 | Method | Results |
| No primer | Brush | Poor adhesion to smooth pluera. |
| | | Good adhesion to peranchaemal |
| | | tissue |
| Saline | Brush | Poor adhesion to smooth pluera. |
| | | Good adhesion to peranchaemal |
| | | tissue |
| Dilute crosslinker | Brush | Excellent adhesion to smooth |
| | | pluera and peranchaemal tissue |
| Acetate buffer | Brush | Excellent adhesion to smooth |
| | | pluera and perancha mal tissue |
| MES buffer | Brush | Excellent adhesion to smooth |
| | | pluera and peranchaemal tissue |
| Glutaraldehyde | Brush | Excellent adhesion to smooth |
| | | pluera and peranchaemal tissue |

1. Saline primer The saline primer was a 0.15M solution of NaCl.
Dilute crosslinker solution A solution of 10% EDC
Buffers: Acetate 0.5M acetate followed by 25 mM acetate both at pH 5.5.
Buffers: MES 0.5M solution of MES at pH 5
Glutaraldehyde 0.5% w/v glutaraldehyde solution

### Example 9

### Effect of Primers on Adhesion to Expanded PTFE

In this experiment expanded PTFE was brush primed using either saline or a 1% w/w perfluorooctanoic acid (PFOA) solution at pH 6.3 and then dried with gauze. sealant consisting of glutaric anhydride derivatized collagen (5.6% w/w), and EDC/Sulfo-NHS (3.8 and 1.5% w/w respectively) at pH 7 was applied. After curing for a total of 3 minutes the adhesion to expanded PTFE examined. The sealant applied to the saline treated expanded PTFE could be pulled off entirely with no cohesive failure. The sealant applied to the PFOA treated expanded PTFE could not be removed without cohesive failure.

### Example 10

### Use of Sealant Compositions

Use of Compositions as a Lung Sealants.

An anesthetized dog was used as an experimental model for thoracic surgical complications including pulmonary leaks.

Wound sites were made at various points on the lobe of the lung using scissors. The wound resulted in bleeding and loss of air. Bleeding was terminated by cauterization and an air leak was confirmed as evidenced by air bubbles from the submerged lung.

In a first experiment, the wound site and surrounding tissue (parenchyma and pluera) were brush primed using 0.5M MES. A device consisting of BSA (35% w/w), tyloxapol (5% w/w), DPPC (5% w/w), and EDC (2% w/w) at a pH of 6.3 was then applied. The sealant viscosity was such that the material easily spread on the lung surface, but did not run off the wound site. After a total curing time of 3 minutes the lung was inflated. No air leaks were observed.

In a second experiment, the wound site and surrounding tissue (parenchyma and pluera) were brush primed using 0.5M MES and the device was then applied. The device consisted of BSA (40% w/w), sodium dodecylsulfate (SDS) and EDC (2% w/w) at a pH of 7. After curing for a total of 7 minutes the lung was inflated. No air leaks were observed.

In one experiment a 40% solution of BSA was prepared by using a 25/10 ratio of BSA and glutaric anhydride derivatized BSA. This solution (pH 6) was used with a 16.67% aqueous solution of EDC.HCl at a ratio of 8:1 (vol/vol) on a porcine lung (ex vivo) to seal a planar wedge resection. The solution on curing adhered to the lung tissue and withstood a static air pressure in excess of 60 mm of Hg (average lung pressure during surgery is in the range of 20-25 mm of Hg).

In another experiment, a 40% (pH 6) BSA solution was mixed with Tyloxapol and dipalmitoyl phosphatidyl choline (DPPC) such that they were 1 mg/ml and 14 mg/ml, respectively. The dispersion was mixed (10:1) with an aqueous solution of EDC HCl (20%) and applied to a porcine lung in a planar wedge resection model. The site was previously primed with a 30% solution of BSA (pH 5.5). The material was allowed to attain maximum strength (about 4-5 minutes), and then tested. The material withstood a dynamic pressure of about 100 mm of Hg before lung tissue rupture occurred.

In another experiment, Gelatin (300 Bloom) was mixed with DPPC and tyloxapol in a similar ratio. The material was a gel at room temperature. The material was warmed to about 45° C. and mixed appropriately with an aqueous EDC.HCl solution. On applying to the lung tissue, the material gelled rapidly and adhered satisfactorily to the wound site.

A preferred sealant composition for lung applications is prepared by mixing a 35% (w/w) of albumin with an EDC cross-linker as described herein.

Use of Compositions as Vascular Sealants

An anesthetized, heparinized, hemodiluted dog was used as an experimental model for vascular surgical complications including anastomoses and suture hole leaks.

Natural-to-synthetic end-to-side anastomoses were created in the femoral and carotid arteries using 6 mm diameter expanded PTFE with suture spacing between 2 and 2.5 mm. The artery was pressurized by removal of clamps and blood leakage was confirmed.

In the first experiment the anastomosis was brush primed using a saline solution to remove excess blood and the cleaned anastomoses was dried with gauze. A device consisting of glutaric anhydride derivatized collagen (5.6% w/w), perflurooctanoic acid (0.5% w/w), and EDC/Sulfo-NHS (3.8 and 1.5% w/w respectively) at pH 7 was applied using an applicator tip. After curing for a total of 3 minutes the anastomosis was pressurized and no blood leakage was observed.

In a second experiment the anastomosis was brush primed using a saline solution to remove excess blood and the cleaned anastomoses was dried with gauze. A device consisting of BSA-perfluorooctanoamide (25% derivatization, 30% w/w), and EDC (2.5% w/w) at pH 6.5 was applied using a splayed applicator tip. After curing for a total of 3 minutes the anastomosis was pressurized and no blood leakage was observed.

In another experiment the anastomosis was brush primed with 0.5 M MES (pH 5). A device consisting of aqueous BSA (36 w.w %), PEG 600 (15 w/w %), PFOA (2.5 w.w %), MES (0.9 w/w %), and EDC (2 w/w %) at pH 6.7 was then applied. After curing for a total of 3 minutes the anastomosis was pressurized and no blood leakage was observed.

In another experiment an anastomosis was brush primed with 0.5 M MES (pH 5). A device consisting of aqueous BSA (40 w/w %), pectin (2.3 w/w %), PFOA (2.8 w/w %), MES (0.9 w/w/%, and EDC (2 w/w %) at pH 6.7 was then applied. After curing for a total of 3 minutes the anastomosis was pressurized and no blood leakage was observed.

A preferred sealant composition for vascular applications is prepared by mixing a 35% (w/w) of albumin with an EDC cross-linker as described herein.

Use of Compositions as Dural Sealants

An anesthetized dog was used as an experimental model for dural surgical complications including cerebrospinal fluid leaks.

Following a craniotomy an incision was made in the dura of about 1 cm in length and leakage of cerebrospinal fluid was detected. Fluid stasis was achieved by tilting the animal and then sealant was applied. A sealant was considered effective if no visible leaking could be detected.

In a first experiment the incision was brush primed using a saline solution to remove excess blood, and the cleaned dura was dried with gauze. A mixed sealant composition consisting of approximately 18% alpha-globulin (w/w) and 2% EDC (w/w) at pH 5.7 was applied to the incision. After curing for a total of 3 minutes the dog was tilted head down and observations made for leaks. No leakage was observed. The dura was further pressurized using saline through a catheter and no leakage was observed.

In a second experiment the incision was spray primed using a 0.25 M MES solution at pH 5 to remove excess blood, and the cleaned dura was dried with gauze. A mixed sealant composition consisting of approximately 18% BSA (w/w), 1.8% alginate (w/w) and 2% EDC (w/w) at pH 5.7 was applied to the incision. After curing for a total of 3 minutes the dog was tilted head down and observations made for leaks. No leakage was observed. The dura was further pressurized using saline through a catheter and no leakage was observed.

A preferred sealant composition for dural applications is prepared by mixing a 20% (w/w) of albumin with an EDC cross-linker as described herein.

Use of Compositions in Seroma Prevention

A preferred sealant composition for seroma prevention is prepared by mixing a 40% (w/w) solution of albumin with an EDC cross-linker as described herein. The resulting sealant forms a strong thin gel that is useful to adhere tissue planes together. This composition is particularly useful to adhere tissue planes together after a surgical removal of tissue matter, for example after a mastectomy.

Use of Compositions for Bonding Tissue

In a preferred embodiment, a composition of the invention is used to bond a first tissue to a second tissue. One or both tissues may be primed as described herein. A composition of the invention may be applied to either one or both of the tissues to be bonded. The first and second tissues are then held together for a sufficient time to allow the cross-linking reaction to create a stable bond or adhesion between the first and second tissues.

### Example 11

### Adhering to End-to-Side Arterial Anastomosis of Expanded PTFE Graft to Artery

In a first experiment, a sealant mixture of BSA and EDC.HCl was delivered in vitro onto an end-to-side anastomosis of an expanded PTFE (expanded polytetrafluoroethylene) graft onto a porcine aorta. The mixture was delivered through a static mixing nozzle, and contained a 9.3:1 ratio of 35% BSA (pH 5.55): 40% EDC.HCl. The adhesive mixture was slowly extruded onto all sides of the anastomosis. The mixture cross-linked fairly rapidly. Indeed, the gel could be neatly cut with scissors ˜30 seconds after its application. The artery was pressurized by introducing saline via a large syringe, and the adhesive treated graft provided a good seal.

In a second experiment, the same composition was used on a 2×20 mm expanded PTFE patch sewn into a porcine carotid artery in vivo. The albumin sealant adhered to the vessel and sealed the leaking suture line.

Use of PTFE Adhesion-Enhancing Additives with Surgical Sealants

A PTFE adhesion-enhancing additive is provided to a sealant consisting of two components A and B. Component A is a mixture of crosslinkable polymer (e.g., protein) and a PTFE adhesion-enhancing additive (e.g., perfluorooctanoic acid). Component B is a solution of crosslinking agent(s). The concentration of component A depends on the protein, the desired handling characteristics, and desired final gel properties. When these two components are mixed the resulting solution is activated and starts to cure. The rate of cure is dependent on the precise composition of the two components and thus provides the ability to slow the cure sufficiently to allow for application followed by a short set time.

In one example, Component A consists of collagen or modified collagen plus a perfluorinated alkanoic acid. As used herein, “modified collagen” is collagen with 10-50 mole % of its accessible primary amines modified with glutaric anhydride. The collagen or modified collagen is at a concentration of from 40 to 100 mg/mL and at a pH of from 5 to 8.5. The perfluorinated compound is at a concentration of from 1 to 20 mg/ml.

Component B comprises from 1 to 100 mg of a carbodiimide and from 1 to 40 mg of N-hydroxysuccinimide dissolved in water. The volume of water used to dissolve the crosslinker is between 1 and 1/10 the volume of component A.

Preferably, component A is a solution of modified collagen (25-40%) at 60-80 mg/mL and with a pH between 6.8 and 7.2 containing 4-6 mg/mL perfluorooctanoic acid. This is mixed with a solution of 25-45 mg of 1-ethyl-3-trimethyl propyl carbodiimide (EDC) and 10-20 mg of sulfo-N-hydroxysuccinimide (sulfo-NHS) dissolved in a volume of water from ½ to ⅔ of the volume of component A.

In one experiment, Component A was prepared by dissolving 600 mg of freeze dried, modified collagen (27% derivatized according to methods known in the art) in 10 ml of an aqueous solution of 160 mM sodium hydroxide. This resulted in slightly viscous solution with a pH of 7.15. 50 mg of perfluorooctanoic acid and were added and mixed until dissolved. The final pH of the solution was 6.9. Addition of the perfluorooctanoic acid caused aeration of the solution. 0.6 cc of aliquots of component A were transferred to 1 cc syringes. The samples were centrifuged to remove air.

In the same experiment, component B was prepared as follows. The crosslinkers (EDC and sulfo-NHS) were stored dry, under nitrogen, in capped vials and reconstituted just prior to use. 37±5 mg EDC and 15+3 mg sulfo-NHS were added to each vial. Each vial was reconstituted with 0.15 cc ultrapure water just prior to mixing with component A.

After reconstitution, component B was drawn into a 1 cc syringe and the air was removed. The syringes containing components A and B were connected by a luer lock connector. The components were mixed for five to ten seconds. The sealant cured to an unworkable gel in 60±10 seconds and was fully cured in 180 seconds.

Evaluation of the Effectiveness of Additives

The effectiveness of an additive can be evaluated by qualitative assessment of the adhesive properties of the resulting mixture. These properties are generally categorized as follows:

Poor No adhesion

Fair Resists pulling off, but adhesion fails

Good Cohesive failure before adhesive

For example, whereas a sealant without an additive such as perfluorooctanoic acid has been found to have “Good” adhesive properties when assayed with tissue but “Poor” when assayed with expanded PTFE, a sealant with perfluorooctanoic acid was found to have “Good” adhesive properties in either assay.

A second testing method involves comparing the contact angle of the solution without the additive to the contact angle observed in the presence of the additive. Generally, smaller contact angles are preferred when increased wetting and adhesion are desired. Greater contact angles are preferred when adhesion is not desired. For example, deionized water has a 119 degree contact angle with PTFE. An aqueous solution of 5% octanoic acid, however, has a 25 degree contact angle. The presence of octanoic acid can therefore improve “wetting” of an aqueous solution on PTFE. Similarly, one mL of a 38% solution of bovine serum albumin in 5% octanoic acid, when crosslinked with 50 mg EDC produces a gel with “Good” adherence to PTFE.

A third testing method involves applying the sealant to end-to-side anastomoses (natural to expanded PTFE), filling the graft with liquid, and subjecting it to pressure. The pressure at which it starts to leak is recorded as its burst pressure. An effective additive will increase the observed burst pressure when used in conjunction with an appropriate sealant and graft material.

The final method of testing is in surgery using dogs. End-to-side anastomoses and suture line models were tested.

In Vivo Testing (Results from Surgery):

| Formulation | |
|---|---|
| (see text below) | Results |
| | All applications are on end-to-side |
| | anastomoses-natural to expanded PTFE- |
| | unless otherwise noted |
| 162-117B | Femoral artery-profuse bleeding before |
| | application. No bleeding after application |
| 162-111B | Carotid artery-suture weeping before |
| | application. No bleeding after application. |
| 162-143A | Femoral artery-profuse bleeding before |
| | application. Stops 95% of bleeding |
| 162-143A | Femoral artery-profuse bleeding before |
| | application. Stops 95% of bleeding. |
| 162-143A | Reapplication on top of previous. 100% of |
| | bleeding stopped. |
| 162-143A | Carotid artery-profuse bleeding before |
| | application. No bleeding after application |
| 162-143A | Carotid artery-profuse bleeding before |
| | application. No bleeding after application |
| 162-143A | Carotid artery-suture line (1 cm, 5 sutures)- |
| | excessive bleeding. 100% of bleeding |
| | stopped. |

162-117B: 0.8 mL of a solution of 80 mg/mL collagen (27% derivatized) and 0.5 mg/mL perfluorooctanoic acid, pH=6.88, were mixed with 0.4 mL of a solution of 125 mg/mL EDC and 37.5 mg/mL sulfoNHS. The vessel was rinsed with saline and dried with gauze. The sealant was applied using a splayed applicator tip.

162-111B: 0.8 mL of a solution of 80 mg/mL collagen (27% derivatized), pH=6.9, were mixed with 0.4 mL of a solution of 125 mg/mL EDC and 37.5 mg/mL sulfoNHS. The vessel was rinsed with a 1% perfluorooctanic acid solution (pH=7) and dried with gauze. The sealant was applied using a splayed applicator tip.

162-143A: 0.6 mL of a solution of 80 mg/mL collagen (27% derivatized), pH=6.9, were mixed with 0.4 mL of a solution of 125 mg/mL EDC, 37.5 mg/mL sulfoNHS, and 10 mg/mL of perfluorooctanoic acid. The vessel was rinsed with saline and dried with gauze. The sealant was applied using a splayed applicator tip.

### Example 12

### Use of Additives to Promote the Adhesion of BSA-Based Sealants to PTFE

Cross-linking experiments with different additives were performed using 30, 33, and 38% (weight/volume) solutions of BSA. Cross-linking was initiated by mixing approximately 10-20 mg of EDC per ml of BSA solution.

The sealant properties of the cross-linked reaction products were evaluated by assessing the adhesion of the cross-linked product to PTFE and vascular tissue.

The addition of octanoic acid (approximately 5% final concentration) resulted in a cross-linked product with good adhesion to PTFE whereas using octanoic acid (approximately 1% final concentration), isopropanol (approximately 20% final concentration), isobutanol (approximately 8% final concentration) as an additive resulted in poor adhesion to PTFE.

These results correlate to average contact angles measured for these additives on PTFE:

| Solvent/Sample | Average Contact Angle (degrees) |
|---|---|
| DIH₂0 | 119 |
| 20% Isopropanol | 95 |
| 8% Isobutanol | 95 |
| 1% Octanoic acid | 88 |
| 5% Octanoic acid | 25 |
| 1% Perfluoro octanoic acid | 75 |
| 1% Perfluoro sebacic acid | 90 |
| 33% BSA/5% Octanoic acid | 24 |

A low contact angle on PTFE for the additive correlates with good adhesion to PTFE.

### Example 13

### Effect of Derivatization of BSA

BSA was derivatized to increase its hydrophobicity with reactive molecules with hydrophobic tails.

In one experiment, 10 g of BSA was dissolved in 200 ml of 0.05N phosphate buffer and pH adjusted to 8.5. 6.89 ml of hexanoic acid anhydride was added in an acetone solution (the acetone solution is saturated with hexanoic acid anhydride) at once. There was no obvious change in the pH of the solution. The reaction was allowed to stir for 2 days at low temperature. The mixture was diafiltered, pH adjusted to 6.0, and dried. Solutions of the derivatized BSA exhibited a more hydrophobic nature as evidenced from contact angle studies.

In one experiment, 10 g of BSA was dissolved in 90 ml of de-ionized water and 100 ml of 0.05N phosphate buffer and pH adjusted to 8.5. 13 g of pyromellitic dianhydride was added in an acetone solution dropwise. The pH was maintained at 8-9 using dilute NaOH. The reaction was allowed to stir overnight at low temperature (approximately 4° C.). The mixture was diafiltered, pH adjusted to 6.0, and dried. Solutions of the derivatized BSA exhibited a more hydrophobic nature as evidenced from contact angle studies. The solution was also highly viscous as compared to BSA solutions of similar concentrations.

In one experiment, 10 g of BSA was dissolved in 67 ml of 0.05N phosphate buffer. The pH of the resulting solution was adjusted to 8.5, and 6 g of tetra fluoro phthalic anhydride was added as a solution in acetone. The pH was maintained at 8-9 using dilute NaOH. After several hours of stirring, the reaction mixture was diafiltered, pH adjusted to 6.0, and dried. Solutions of the derivatized BSA exhibited a more hydrophobic nature as evidenced from contact angle studies.

In one experiment, 20 g of BSA was dissolved in 500 ml of a 65/35 mixture of de-ionized water and methanol. The pH of the pale yellow solution was adjusted to 9.0, and 8 ml of (2, 2, 3, 3, 4, 4, 5, 5, 6, 6, 7, 7, 8, 8, 9, 9, 9-heptadecafluorononyl)-oxirane was added all at once in a 50% acetone solution. The reaction was allowed to stir for 2 days, maintaining the pH at 9. The slightly turbid solution was centrifuged, dialyzed, pH adjusted to 6.0, and allowed to dry. Solutions of the modified BSA solution exhibited higher viscosity and an improved wettability towards expanded PTFE graft, and on cross-linking with the appropriate amount of EDC.HCl, adhered very well to the graft and natural tissue.

### Example 14

### Effect of Partially Cross-Linked Protein on Sealant Viscosity

The viscosity of a sealant comprising bovine serum albumin (BSA), tyloxapol, and dipalmitoyl-phosphatidyl choline was modified by replacing bovine serum albumin with partially cross-linked bovine serum albumin. The partially cross-linked albumin samples were prepared as follows: A 40% w/w BSA solution (pH 6.85) and a 4% w/w EDC solution were mixed at room temperature at a ratio of 9 parts BSA solution to 1 part EDC solution. The solutions were stirred for set periods to allow the viscosity to build and then the reaction was quenched by diluting by four volumes of water and adjusting the pH of the resulting solution to 10. The resulting solution was purified by exhaustive dialysis and lyophillized. The resulting products were formulated at the concentrations described above and viscosity was measured using a Brookfield cone and plate viscometer (spindle number S52, 25° C., 20PRM).

The following table outlines the results:

| Sample Description | Viscosity (cps) 1 |
|---|---|
| Control (normal albumin) | 427 |
| Reaction time: 20 minutes | 970 |
| Reaction Time: 24 minutes | 1300 |
| Reaction Time: 32 minutes | 2900 |

1-167. (Cancelled).

168. A tissue sealant or adhesive, comprising:
(a) a first solution comprising a polymer, the first solution having a first pH value; and
(b) a second solution comprising a cross-linker, the second solution having a second pH value,
wherein the cross-linker is activated at a pH between the pH of the first solution and the pH of the second solution.

169. The tissue sealant or adhesive according to claim 168 wherein the first solution has a pH between about 3.0 and about 6.0.

170. The tissue sealant or adhesive according to claim 168 wherein the second solution has a pH between about 7.0 and about 10.0.

171. The tissue sealant or adhesive according to claim 168 wherein the polymer comprises a protein.

172. The tissue sealant or adhesive according to claim 171 wherein the protein is selected from the group consisting of albumin, collagen, gelatin, globulin, elastin, protamine, and histone.

173. The tissue sealant or adhesive according to claim 168 wherein the polymer comprises albumin.

174. The tissue sealant or adhesive according to claim 168 wherein the second solution further comprises a protein.

175. A method of forming a tissue sealant or adhesive comprising:
(a) providing a first solution comprising a polymer, the first solution having a first pH value; and
a second solution comprising a cross-linker, the second solution having a second pH value,
wherein the cross-linker is activated at a pH between the pH of the first solution and the pH of the second solution;
(b) mixing the first solution and the second solution, wherein the cross-linking agent is activated upon mixing of the first solution with the second solution.

176. The method of claim 175 wherein the first solution and the second solution are mixed prior to application to a tissue locus.

177. The method of claim 175 wherein the first solution and the second solution are mixed after application to a tissue locus.
